(19) United States
(12) Patent Application Publication
(10) Pub. No.: US 2012/0202819 A1
(43) Pub. Date: Aug. 9, 2012

(54) COMBINATION THERAPY USING A BETA 3 ADRENERGIC RECEPTOR AGONISTS AND AN ANTIMUSCARINIC AGENT

(75) Inventors: Scott D. Edmondson, Clark, NJ (US); Mary Struthers SinhaRroy, Edison, NJ (US); Hiroshi Nagabukuro, Natick, MA (US); William S. Denney, Somerville, MA (US); Tara F. Frenki, Blue Bell, PA (US)

(73) Assignee: Merck Sharp & Dohme Corporation, Rahway, NJ (US)

(21) Appl. No.: 13/500,574

(22) PCT Filed: Sep. 27, 2010
(86) PCT No.: PCT/US10/50328

§ 371 Date: Apr. 5, 2012

Related U.S. Application Data
(60) Provisional application No. 61/249,386, filed on Oct. 7, 2009.

(51) Int. Cl. A61K 31/427; A61K 31/517; A61K 31/501; A61K 31/502; A61K 31/513; A61P 13/10; A61K 31/4196; A61K 31/437; A61K 31/519; A61K 31/4725; A61K 31/46; A61K 31/445; A61K 31/428; A61K 31/4439

(52) U.S. Cl. 514/248; 514/370; 514/367; 514/266.2; 514/252.05; 514/274; 514/269; 514/343; 514/383; 514/299; 514/259.5; 514/305; 514/278; 514/327

(57) ABSTRACT

Described herein is an improved method of treating overactive bladder, wherein the method comprises administering to a patient in need thereof a beta 3 adrenergic receptor agonist, an antimuscarinic agent, and an optional selective M₂ antagonist. Such combination therapy provides improved efficacy and/or reduced side effects.

### BACKGROUND OF THE INVENTION

The function of the lower urinary tract is to store and periodically release urine. This requires the orchestration of storage and micturition reflexes which involve a variety of afferent and efferent neural pathways, leading to modulation of central and peripheral neuroeffector mechanisms, and resultant coordinated regulation of sympathetic and parasympathetic components of the autonomic nervous system as well as somatic motor pathways. These proximally regulate the contractile state of bladder (detrusor) and urethral smooth muscle, and urethral sphincter striated muscle.

Overactive bladder (OAB) is characterized by the symptoms of urinary urgency, with or without urgency urinary incontinence, usually associated with frequency and nocturia. The prevalence of OAB in the United States and Europe has been estimated at 16 to 17% in both women and men over the age of 18 years. Overactive bladder is most often classified as idiopathic, but can also be secondary to neurological condition, bladder outlet obstruction, and other causes. From a pathophysiologic perspective, the overactive bladder symptom complex, especially when associated with urge incontinence, is suggestive of detrusor overactivity. Urgency with or without incontinence has been shown to negatively impact both social and medical well-being, and represents a significant burden in terms of annual direct and indirect healthcare expenditures.

Anti-muscarinic agents have been used for treating incontinence conditions such as OAB. For example, tolterodine, or (R)-N,N-diisopropyl-3-(2-hydroxy -5-methylphenyl)-3-phenylpropanamine, has been marketed for the treatment of urge incontinence and other symptoms of unstable or overactive urinary bladder. Both tolterodine and its major active metabolite, the 5-hydroxymethyl derivative of tolterodine, are thought to contribute to the therapeutic effect. However, current medical therapy for OAB using anti-muscarinic agents often is suboptimal, as many patients either do not demonstrate an adequate response to current treatments, and/or are unable to tolerate the considerable side effects such as dry mouth with the current treatments.

Therefore, there is a continuing need for improved therapies that provide more effective treatment of OAB and/or reduced side effects.

### BRIEF DESCRIPTION OF THE DRAWINGS

FIG. 1 (FIG. 1) is a diagram showing the isobologram analysis.

FIG. 2 (FIG. 2) is a diagram showing the isobolograms of the inhibition of detrusor contraction induced by the combinations of CL316243 with tolterodine (A), oxybutynin (B) or darifenacin (C).

FIG. 3 (FIG. 3) is a diagram showing the isobolograms of the inhibition of detrusor contraction induced by the combinations of Compound 12, and tolterodine (A) and darifenacin (B).

FIG. 4 (FIG. 4) is a diagram showing CL316243 with or without pre-treatment of methoctramine.

FIG. 5 (FIG. 5) is a diagram showing the isobolograms of the inhibition of detrusor contraction induced by the combinations of CL316243 and darifenacin with (A) or without (B) a pretreatment of methoctramine.

FIG. 6 (FIG. 6) is a diagram showing the isobolograms of the inhibition of detrusor contraction induced by the combinations of CL316243 and oxybutynin at different ratios.

### SUMMARY OF THE INVENTION

It has now surprisingly been found that a combination therapy using a beta 3 adrenergic receptor agonist (hereinafter, “β3-AR agonist”), an antimuscarinic agent, and an optional selective M₂ antagonist provides synergistic effect for treating overactive bladder. Combination compositions comprising a β3-AR agonist, an antimuscarinic agent and an optional selective M₂ antagonist are also described.

### DESCRIPTION OF THE INVENTION

Described herein is a method of treating overactive bladder, wherein the method comprises administering to a patient in need thereof a _{33-AR} agonist, an antimuscarinic agent, and an optional selective M₂ antagonist. Such combination therapy provides synergistic effect and thus improved efficacy and/or reduced side effects.

It has now surprisingly been found that the M₂ antagonism of an antimuscarinic agent may play an important role in providing synergy for treating OAB in a combination therapy comprising a β3-AR agonist and the antimuscarinic agent. While not wishing to be bound by theory, it is generally believed that the M3 antagonism of an antimuscarinic agent is important for OAB efficacy (see, for example, Abrams and Andersson. BJU Int, 100, 987-1006 (2007)). It has now been found that the M₂ antagonism, working together with the M3 antagonism and a β3-AR agonist, provides synergy.

In one embodiment, a synergistic effect is obtained in a combination therapy comprising a β3-AR agonist and an antimuscarinic agent wherein the antimuscarinic agent has an M₂/M₃ ratio of less than about 40. In another embodiment, the antimuscarinic agent has an M₂/M₃ ratio of less than about 20.

Additionally, in a case where an antimuscarinic agent has an M₂/M₃ ratio of greater than 40, synergy may be obtained by using an additional selective M₂ antagonist in a combination therapy comprising a β3-AR agonist and the antimuscarinic agent.

As used herein, the term “synergy” or “synergistic effect” is used to describe a situation where the combined effect of two or more active agents is greater than the sum of the individual active agents. In other words, two or more active agents can interact in ways that the presence of one active agent enhances or magnifies the effects of the second. In contrast, where the combined effect of two or more active agents substantially equals to the sum of the individual active agents, the combined effect is simply additive, but not synergistic. And where the combined effect of two or more active agents is less than the sum of the individual active agents, the combined effect is sub-additive, also not synergistic.

In one embodiment, the combination therapy comprises administering to a patient in need thereof a β3-AR agonist and an antimuscarinic agent, wherein the antimuscarinic agent has an M₂/M₃ ratio of less than about 40. In another embodiment, the antimuscarinic agent has an M₂/M₃ ratio of less than about 30. In another embodiment, the antimuscarinic agent has an M₂/M₃ ratio of less than about 20. In another embodiment, the antimuscarinic agent has an

M₂/M₃ ratio of less than about 15. In yet another embodiment, the antimuscarinic agent has an M₂/M₃ ratio of less than about 10. In still another embodiment, the antimuscarinic agent has an Mill% ratio of about 1.

In another embodiment, the antimuscarinic agent in the combination therapy has an M₂/M₃ ratio of greater than about 0.1. In another embodiment, the antimuscarinic agent has an M₂/M₃ ratio of greater than about 0.5. In another embodiment, the antimuscarinic agent has an M₂/M₃ ratio of greater than about 0.8.

In another embodiment, the antimuscarinic agent in the combination therapy has an M₂/M₃ ratio of from about 0.1 to about 40. In another embodiment, the antimuscarinic agent has an M₂/M₃ ratio of from about 0.5 to about 30. In another embodiment, the antimuscarinic agent has an M₂/M₃ ratio of from about 0.8 to about 20. In another embodiment, the antimuscarinic agent has an M₂/M₃ ratio of from about 1 to about 20. In yet another embodiment, the antimuscarinic agent has an M₂/M₃ ratio of from about 1 to about 15. In still another embodiment, the antimuscarinic agent has an M₂/1v1₃ ratio of from about 1 to about 10.

In one embodiment, the M₂/M₃ ratio is measured using the receptor binding assays described in Ohtake et al. (Biol. Pharm. Bull. 30, 54-58, 2007), which is incorporated herein by reference in its entirety. In another embodiment, the M₂/M₃ ratio is measured using the assays described in Hegde et al. (Curr Opin Invest Drugs. 5, 40-49 (2004), which is incorporated herein by reference in its entirety.

The M₁-M₄ activities of some exemplary antimuscarinic agents reported in Ohtake et al. (Biol. Pharm. Bull. 30, 54-58 (2007)) are shown in Table 1.

TABLE 1
M₁-M₄ Activities of Some Exemplary Antimuscarinic
Agents-Ohtake et al.

| Antimus- | Ki (nM) | | | | |
|---|---|---|---|---|---|
| carinic | | | | | M₂/M₃ |
| Agent | M₁ | M₂ | M₃ | M₄ | ratio |
| Tolterodine | 2.7 ± 0.23 | 4.2 ± 0.51 | 4.4 ± 0.45 | 6.6 ± 1.7 | 1 |
| Oxybutynin | 6.1 ± 1.5 | 21 ± 3.6 | 3.4 ± 0.65 | 6.6 ± 2.7 | 6 |
| Darifenacin | 31 ± 2.6 | 100 ± 14 | 2.0 ± 0.21 | 52 ± 15 | 50 |
| Solifenacin | 26 ± 2 | 170 ± 37 | 12 ± 4.4 | 110 ± 45 | 14 |
| Propiverine | 490 ± 110 | 1400 ± 220 | 350 ± 53 | 900 ± 200 | 4 |

Hegde et al. (Curr Opin Invest Drugs. 5, 40-49 (2004)) described M₁-M₄ activities of some antimuscarinic agents and the M₁-M₄ activities of trospium are shown in Table 2.

TABLE 2
M₂ and M₃ Activities of Trospium-Hegde
et al.

| | Ki (nM) | | | | |
|---|---|---|---|---|---|
| Antimuscarinic | | | | | M₂/M₃ |
| Agent | M₁ | M₂ | M₃ | M₄ | ratio |
| Trospium | 0.75 | 0.65 | 0.5 | 1 | 1 |

Suitable anti-muscarinic agents for the combination therapy include, but are not limited to: tolterodine, oxybutynin (including S-oxybutynin), hyoscyamine, propantheline, propiverine, trospium (including trospium chloride), solifenacin, darifenacin, dicyclomine, ipratropium, oxytrol, imidafenacin, fesoterodine, temiverine, SVT-40776, 202405 by GlaxoSmithKline, TD6301, RBX9841, DDP200, and PLD179.

In one embodiment, the anti-muscarinic agent is selected from the group consisting of tolterodine, fesoterodine, oxybutynin, solifenacin, propiverin, trospium, imidafenacin, and TD63 01. In one embodiment, the M₂/M₃ ratio of the suitable anti-muscarinic agent is less than 40. In another embodiment, the M₂/M₃ ratio is less than 30. In another embodiment, the M₂/M₃ ratio is less than 20. In yet another embodiment, the M₂/M₃ ratio is less than 15. In one embodiment, the M₂/M₃ ratio is measured using the binding assays described in Ohtake et al.

In another embodiment, the anti-muscarinic agent is selected from the group consisting of: tolterodine, fesoterodine, oxybutynin, solifenacin, propiverine, and trospium.

In another embodiment, the anti-muscarinic agent is selected from the group consisting of: tolterodine and oxybutynin.

In yet another embodiment, the anti-muscarinic agent is tolterodine.

Suitable β3-AR agonists include, but are not limited to, CL316243, and compounds shown in Table 3.

TABLE 3
Suitable β₃-AR agonists for combination therapy.

| Compound # | Structure |
|---|---|
| 1 | 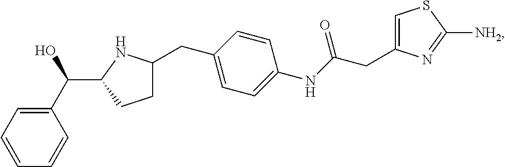 |
| 2 | 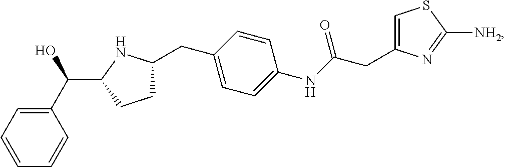 |
| 3 | 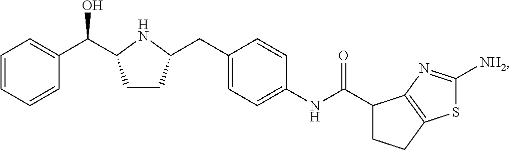 |
| 4 | 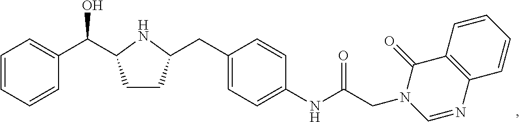 |
| 5 | 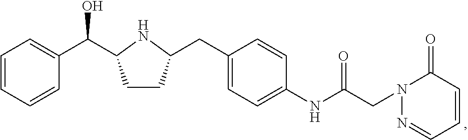 |
| 6 | 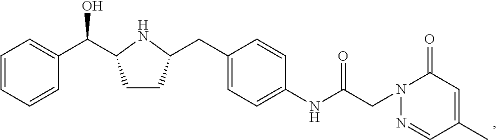 |
| 7 | 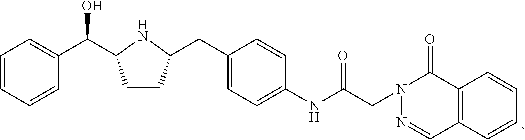 |
| 8 | 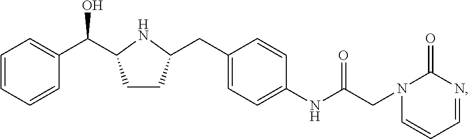 |
| 9 | 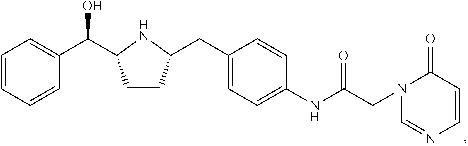 |
| 10 | 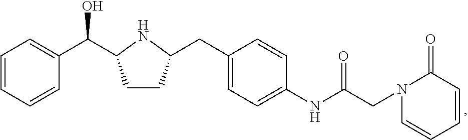 |
| 11 | 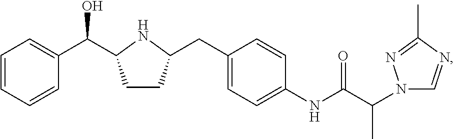 |
| 12 | 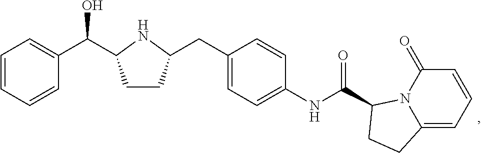 |
| 13 | 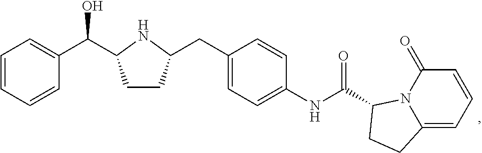 |
| 14 | 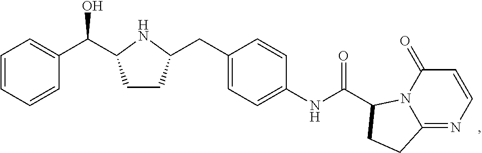 |
| 15 | 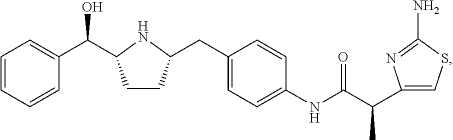 |
| 16 | 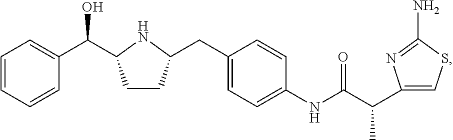 |
| 17 | 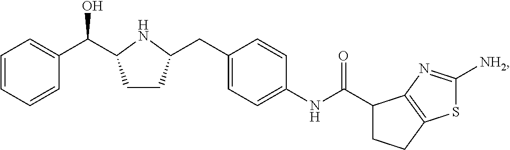 |
| 18 | 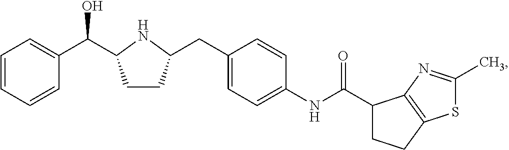 |
| 19 | 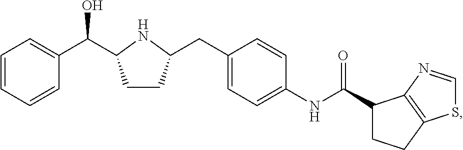 |
| 20 | 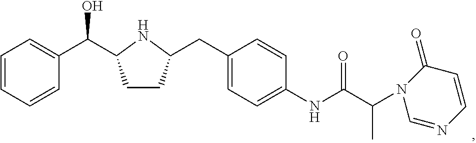 |
| 21 | 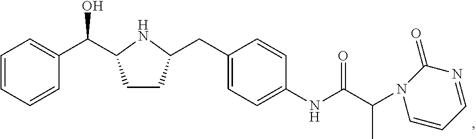 |
| 22 | 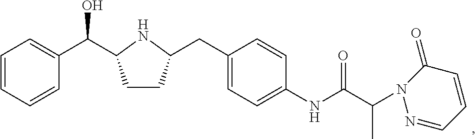 |
| 23 | 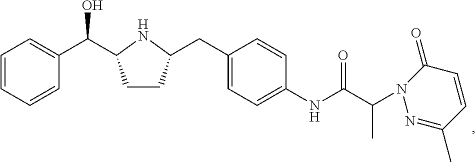 |
| 24 | 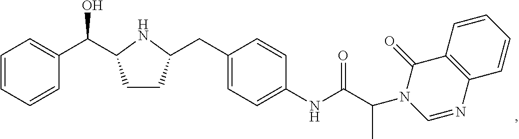 |
| 25 | 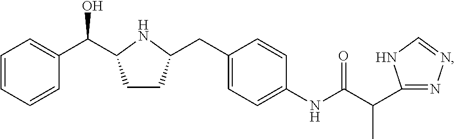 |
| 26 | 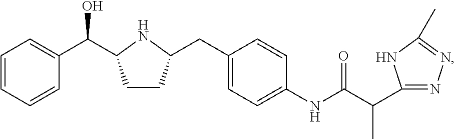 |
| 27 | 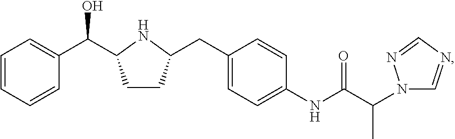 |
| 28 | 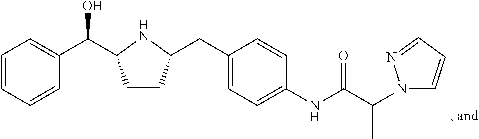 |
| 29 | 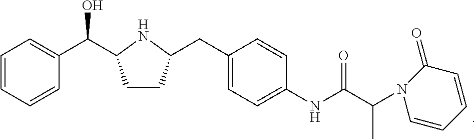 |

In another embodiment, the β3-AR agonist is selected from the compounds listed in Table 4:

TABLE 4
Suitable β₃-AR agonists for combination therapy.

| Compound # | Structure |
|---|---|
| 11 | 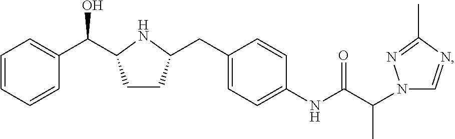 |
| 12 | 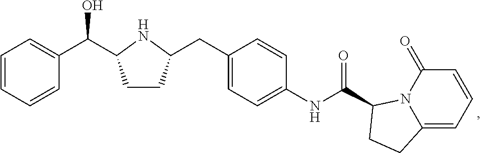 |
| 13 | 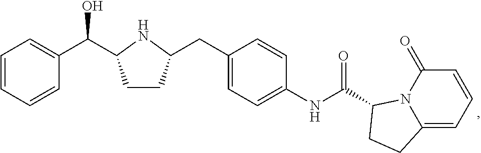 |
| 14 | 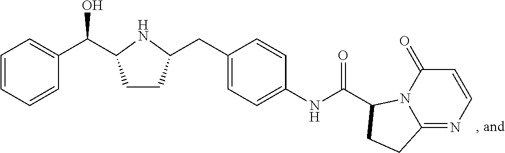 |
| 26 | 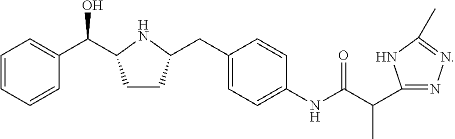 |

In another embodiment, the β3-AR agoinst is selected from the group consisting of

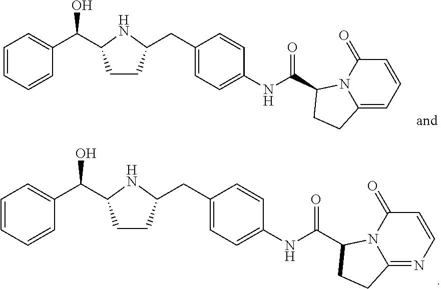

In yet another embodiment, the β3-AR agonist is

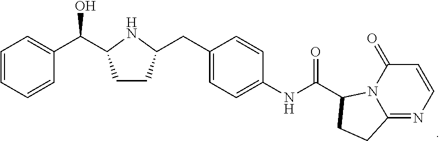

Compounds in Table 3 can be prepared using procedures as described below.

Throughout the application, the following terms have the indicated meanings unless otherwise noted:

| Term | Meaning |
|---|---|
| Ac | Acyl (CH₃C(O)—) |
| Aq. | Aqueous |
| Bn | Benzyl |
| BOC (Boc) | t-Butyloxycarbonyl |
| BOP | Benzotriazol-1-yloxytris(dimethylamino)- |
| | phosphonium hexafluorophosphate |
| ° C. | Degree Celsius |
| Calc. or calc'd | Calculated |
| Celite | Celite ™ diatomaceous earth |
| DCC | Dicyclohexylcarbodiimide |
| DCM | Dichloromethane |
| DIEA | N,N-diisopropyl-ethylamine |
| DMAP | 4-Dimethylaminopyridine |
| DMF | N,N-dimethylformamide |
| EDC | 1-Ethyl-3-(3-dimethylaminopropyl) |
| | carbodiimide |
| Eq. or equiv. | Equivalent(s) |
| ES-MS and | Electron spray ion-mass spectroscopy |
| ESI-MS | |
| Et | Ethyl |
| EtOAc | Ethyl acetate |
| g | Gram(s) |
| h or hr | Hour(s) |
| HATU | O-(7-azabenzotriazol-1-yl)-N, N, N′, N′- |
| | tetramethyluronium hexafluorophosphate |
| HOAc | Acetic acid |
| HOAT | 1-Hydroxy-7-azabenzotriazole |
| HOBT | 1-Hydroxybenzotriazole |
| HPLC | High performance liquid chromatography |
| LC/MS or | Liquid chromatography mass spectrum |
| LC-MASS | |
| L | Liter(s) |
| M | Molar(s) |
| Me | Methyl |
| MeOH | Methanol |
| MF | Molecular formula |
| min | Minute(s) |
| mg | Milligram(s) |
| mL | Milliliter(s) |
| mmol | Millimole(s) |
| MOZ (Moz) | p-Methoxybenzyloxycarbonyl |
| MP | Melting point |
| MS | Mass spectrum |
| nM | Nanomolar |
| OTf | Trifluoromethanesulfonyl |
| Ph | Phenyl |
| Prep. | Preparative |
| Ref. | Reference |
| r.t. or rt | Room temperature |
| Sat. | Saturated |
| SCF CO₂S | Super critical fluid carbon dioxide |
| TBAF | Tetrabutylammonium fluoride |
| TBAI | Tetrabutylammonium iodide |
| TBDPS | Tert-butyl diphenylsilyl |
| TBS, TBDMS | Tert-butyl dimethylsilyl |
| TEA or Et₃N | Triethylamine |
| Tf | Triflate or trifluoromethanesulfonate |
| TFA | Trifluoroacetic acid |
| THF | Tetrahydrofuran |
| TLC | Thin-layer chromatography |
| TMS | Trimethylsilyl |

### Intermediate I

Benzyl [3-(2-oxobut-3-en-1-yl)phenyl] carbamate (i-1):

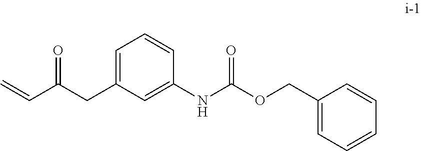

Step A: Ethyl (3-{[(benzyloxy)carbonyl]amino}phenyl) acetate

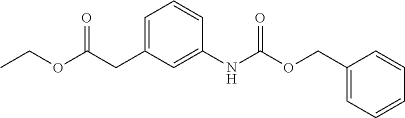

To a solution of methyl (3-aminophenyl) acetate (25 g, 140 mmol) in 250 mL anhydrous DCM was added DIEA (28.5 mL, 155 mmol) and the resulting solution cooled to 0° C. and set under nitrogen atmosphere. To this cooled solution was then added benzyl chlorofounate (21.1 mL, 148 mmol) and the resulting mixture stirred overnight allowing to warm to room temperature. The reaction was washed with 1 M HCl, water, and then brine. The organic layer was dried over sodium sulfate, filtered and concentrated under vacuum. No further purification was necessary and the material (44 g, 99%) was used as is for the next step reaction. LC-MS: m/z (ES) 314 (MH)⁺, 336 (MNa)⁺.

Step B: 3-{[(Benzyloxy)carbonyl]amino}phenyl) acetic acid

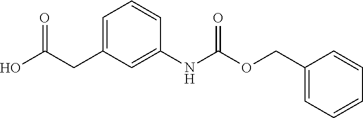

To a solution of 44.0 g (140 mmol) of ethyl (3-{[(benzyloxy)carbonyl]amino}phenyl) acetate) (from Step A) in THF, ethanol, and water (1:1:1, 1500 mL) was added solid LiOH (16.8 g, 700 mmol) and the resulting solution heated. to 60° C. via oil bath for 3 h. The mixture was cooled to room temperature overnight and then 40 mL of concentrate HCl was slowly added, keeping the temperature below 25° C., until the solution was about pH of 2-3. Extract with ethyl acetate (3×750 mL) and then combine and wash organics with water and then brine. Dry organics over sodium sulfate, filter and concentrate under vacuum. The title compound (24.7 g, 87%) was used for the next step reaction without further purification. LC-MS: m/z (ES) 286 (MH)⁺, 308 (MNa)⁺.

Step C: Benzyl (3-{2-[methoxy(methyl)amino]-2-oxoethyl}phenyl)carbamate

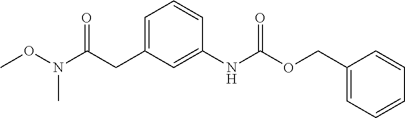

To a suspension of 24.7 g (87 mmol) of (3-{[(benzyloxy)earbonyl]amino}phenyl) acetic acid in 200 mL of dichloromethane (from Step B) was added triethylamine (30.2 mL, 173 mmol) which resulted in some exotherming (+5° C.) and the suspension becoming a solution. After 10 min cooling, HOBt (13.2 g, 87 mmol), N,O-dimethylhydroxylamine HCl (8.5 g, 87 mmol) was added to the solution followed by EDC (16.6 g, 87 mmol) and the resulting mixture stirred at room temperature overnight under nitrogen atmosphere. The solution was transferred to a separatory funnel and washed with 1 M HCl which caused an emulsion. Methanol was added to break up the emulsion and the aqueous was partitioned off. The organics were dried over sodium sulfate, filtered and concentrated under vacuum. Recrystallization of the residue from 1000 mL of 70% hexane in ethyl acetate (heated to reflux and then cooled to room temperature overnight) afforded the title compound (21 g, 74%) as a white solid. LC-MS: m/z (ES) 329 (MH)⁺.

Step D: Benzyl [3-(2-oxobut-3-en-1-yl)phenyl] carbamate (i-1)

To a solution of 15 g (45.7 mmol) of benzyl (3-{2-[methoxy(methyl)amino]-2-oxoethyl}phenyl)carbamate (from Step C) in 1000 mL anhydrous THF under nitrogen atmosphere cooled to 0° C. via ice/water bath was added dropwise via cannula a 1.0 M solution of vinyl magnesium bromide (100 mL in THF, 100 mmol) and the resulting solution stirred for 1 h at 0° C. The reaction was quenched by a slow addition of 500 mL 1 M HCl keeping the temperature below 5° C. and stirred for 30 min. The mixture was then extracted with ethyl acetate and the combined organics washed with water followed by brine. The organics were then dried over sodium sulfate, filtered, and concentrate under vacuum. The residue was purified by Biotage 75M flash eluting with 30% ethyl acetate in hexane to afford the title compound (11 g, 78%) as a light yellow solid. LC-MS: m/z (ES) 310 (MH)⁺, 332 (MNa)⁺. ¹HNMR (500 MHz, CDCl₃) δ:7.44-736 (m, 7H), 7.18 (d, J=8.4 Hz, 2H), 6.70 (br s, 114), 6.44 (dd, J=10.5, 17.6 Hz, 1H), 6.32 (dd, J=1.1, 17.6 Hz, 1H), 5.85 (dd, J=1.1, 10.5 Hz, 1H), 5.22 (s, 2H), 3.86 (s, 2H).

### Intermediate 2

((1 R)-1-[(R)-{[tert-butyl(dimethyl)silyl]oxy)(3-chlorophenyl)methyl]prop-2-en-1-yl}carbamate (i-2)

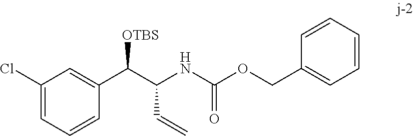

Step A: 1-(3-Chlorophenyl)pron-2-en-1-ol

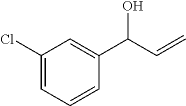

To a cooled solution of 3-chlorobenzaldehyde (22.5 g, 160 mmol) in 100 mL anhydrous THF under inert nitrogen atmosphere was added slowly via syringe a 1.6 M solution of vinyl magnesium chloride in THF (100 mL, 160 mmol) and the solution stirred for three h allowing to warm to room temperature. The reaction was quenched with saturated solution of ammonium chloride and the organic layer was separated, extracted with ethyl acetate (2×200 mL), and organic layers were combined, dried over magnesium sulfate, filtered and concentrated under vacuum. Purification by Horizon MPLC with a 40M+ silica gel column using a gradient eluent of 0-40% ethyl acetate in hexane afforded the title compound (22.4 g, 44%). m/z (ES) 168, 170 (M, M+2)⁺, 190, 192 (MNa, MNa+2)⁺. ¹HNMR (500 MHz, CDCl₃) δ: 7.38 (s, 1H), 7.35-7.22 (m, 3H), 5.90 (ddd, J=7.3, 10.0, 17.4 Hz, 1H), 5.38 (d, J=17.5 Hz, 1H), 5.18 (d, J=7.2 Hz, 1H), 5.15 (d, J=10.1 Hz, 1H), 0.96 (s, 9H), 0.18 (s, 3H), 0.08 (s, 3H).

Step B: Tert-butyl {[1-(3-chlorophenyl)prop-2-en-1-yl]oxy}dimethylsilane

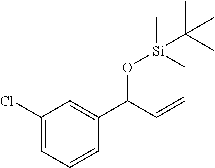

To a solution of 22.4 g (133 mmol) of 1-(3-chlorophenyl)prop-2-en-1-ol in 90 mL anhydrous DMF (from Step A) was added t-butyldimethylsilyl chloride (20.0 g, 133 mmol) and imidazole (18.1 g, 266 mmol) and the resulting solution was stirred overnight under nitrogen at room temperature. Wash with water and extract with ethyl acetate. Separate organics, dry over magnesium sulfate, filter, and concentrate under vacuum. The residue was purified by flash silica gel column eluting with a gradient eluent of 0-15% ethyl acetate in hexane to afford the title compound (16.6 g, 46%). m/z (ES) 282, 284 (M, M+2)⁺; 151, 153 (M-OTBS, M-OTBS+2)⁺.

Step C: {[Text-butyl (dimethypsilyl]oxy}(3-chlorophenyl)acetaldehyde

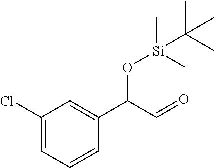

To a solution of 4.0 g (14.2 mmol) of tert-butyl {[1-(3-chlorophenyl)prop-2-en-1-yl]oxy}dimethylsilane in dichloromethane cooled to −78° C. via dry ice/acetone bath (from Step B) was bubbled ozone until the solution maintained a slight blue color. Nitrogen gas was then bubbled into the solution until it turned clear. Methyl sulfide was added to the solution and the resulting mixture was allowed to stir overnight at room temperature. The material was concentrated under vacuum and the residue purified via Horizon MPLC with a 40M+ silica gel column, eluting with a gradient eluent of 0-50% ethyl acetate in hexane to afford the product (3.57 g, 89%).

Step D: N-[(1E)-2-{[tert-butyl(dimethyl)silyl]oxy}-2-(3-chlorophenypethylidene]-2-methylpropane-2-sulfinamide

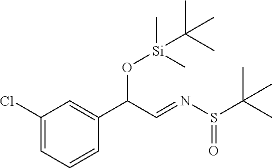

To a solution of 3.0 g (10.6 mmol) of {[teat-butyl (dimethypsilyl]oxy}(3-chlorophenyl)acetaldehyde (from Step C) and 1.3 g (10.6 mmol) of (R or S)-2-methyl-2-propanesulfinamide in 50 mL anhydrous dichloromethane was added copper(II) sulfate (3.4 g, 21.2 mmol) and the resulting mixture was stirred at room temperature under nitrogen atmosphere for 16 h. Wash reaction with water and extract with dichloromethane. Dry the organics with magnesium sulfate, filter and concentrate under vacuum. The residue was purified by Horizon MPLC, with a 40M+ silica gel column, eluting with a gradient eluent system of 0-25% ethyl acetate in hexane to afford the title compound (3.26 g, 80%). %). m/z (ES) 387, 390 (M, M+2)⁺.

Step E: N-{1-[{[tert-butyl(dimethyl)silyl]oxy}(3-chlorophenyl)methyl]-prop-2-en-1-yl}2-methylpropane-2-sulfinamide

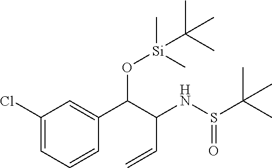

To a solution of 2,4 g (6.20 mmol) of N-[(1E)-2-{[teat-butyl(dimethyl)silyl]oxy}-2-(3-chlorophenyeethylidene]-2-methylpropane-2-sulfinamide in 20 mL anhydrous THF cooled to 0° C. under nitrogen atmosphere (from Step D) was added a 1.6 M solution of vinyl magnesium chloride in THF (3.90 mL, 6.2 mmol) via syringe and the resulting mixture stirred for 1 h. The mixture was allowed to warm to room temperate and stirred for an additional hour. The reaction was quenched with saturated solution of ammonium chloride and extract with ethyl acetate. Combine organics, dry over magnesium sulfate, filter and concentrate under vacuum. The residue was purified by Horizon MPLC, with a 40M+ silica gel column, eluting with a gradient eluent system of 0-35% ethyl acetate in hexane to afford all four diastereomers as single isomers.

By NMR the four products obtained were diastereomers of each other. The isomers were labeled as they eluted off the silica gel column. The first isomer that eluted off was named isomer 1 and then isomers 2, 3 and lastly isomer 4.

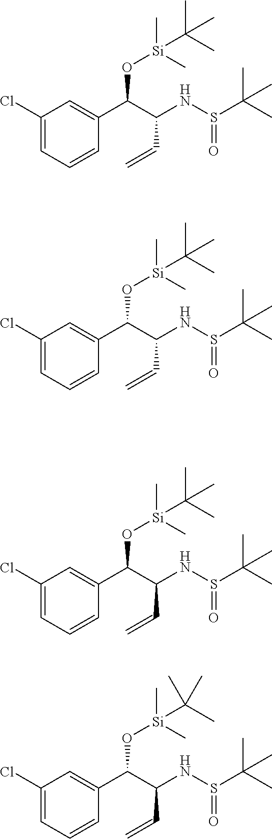

Isomer 1: m/z (ES) 416, 418 (M, M+2)⁺, 438, 440 (MNa, MNa+2)⁺. ¹HNMR (500 MHz, CDCl₃) δ:7.32 (s, 1H), 7.30 (br d, J=7.5, 1H), 7.26 (br d, J=6.2 Hz, 2H), 7.22-7.18 (m, 1H), 5.60 (ddd, J=7.3, 10.3, 17.4 Hz, 1H), 5.15 (d, J=10.3 Hz, 1H), 5.00 (d, J=17.3 Hz, 1H), 4.57 (d, J=7.4 Hz, 1H), 3.98-3.94 (m, 2H), 1.64 (br s, 1H), 1.23 (s, 9H), 0.91 (s, 9H), 0.08 (s, 3H), -0.18 (s, 3H).
Isomer 2: m/z (ES) 416, 418 (M, M+2)⁺, 438, 440 (MNa, MNa+2)⁺. ¹HNMR (500 MHz, CDCl₃) δ: 7.33-7.31 (m, 2H), 7.26 (br d, J=5.0 Hz, 2H), 7.20-7.16 (m, 1H), 5.44 (ddd, J=7.2 , 10.0, 17.4 Hz, 1H), 5.26 (overlapping d, J=7.3 Hz, 1H), 5.25 (overlapping d, J=17.3 Hz, 1H), 4.84 (d, J=4.4 Hz, 1H), 4.02(dt, J=4.4, 7.8 Hz, 1H), 3.80 (d, J=4.4 Hz, 1H), 1.20 (s, 9H), 0.94 (s, 9H), 0.14 (s, 3H), −0.12 (s, 3H).
Isomer 3: m/z (ES) 416, 418 (M, M+2)⁺, 438, 440 (MNa, MNa+2)⁺. ¹HNMR (500 MHz, CDCl₃) δ: 7.32-7.29 (m, 2H), 7.26-7.24 (m, 2H), 7.22-7.20 (m, 1H), 6.04 (ddd, J=7.1, 10.4, 17.4 Hz, 1H), 5.40 (d, J=10.2 Hz, 1H), 5.32 (d, J=17.3 Hz, 1H), 4.80 (d, J=4.0 Hz, 1H), 3.88-3.80 (m, 1H), 3.55 (d, J=9.4 Hz, 1H), 1.09 (s, 9H), 0.95 (s, 9H), 0.09 (s, 3H), −0.10 (s, 3H).
Isomer 4: m/z (ES) 416, 418 (M, M+2)⁺, 438, 440 (MNa, MNa+2)⁺. ¹HNMR (500 MHz, CDCl₃) δ: 7.32 (s, 1H), 7.30 (br d, J=7.5, 1H), 7.27-7.25 (m, 2H), 7.21-7.18 (m, 1H), 5.92 (ddd, J=7.1, 10.3, 17.4 Hz, 1H), 5.23 (d, J=10.4 Hz, 1H), 5.18 (d, J=17.4 Hz, 1H), 4.75 (d, J=4.2 Hz, 1H), 3.88-3.82 (m, 1H), 3.33 (d, J×9.4 Hz, 1H), 1.19 (s, 9H), 0.94 (s, 9H), 0.09 (s, 3H), −0.14 (s, 3H).
   Step F: ((1R)-1-[(R)-{[tert-butyl(dimethypsilyl]oxy)(3-chlorophenyl)methyl]prop-2-en-1-yl}carbamate (i-2)

To isomer 1 (510 mg, 2.22 mmol) of N-{1-[{[tert-butyl(dimethyl)silyl]oxy}(3-chlorophenyl)methyl]-prop-2-en-1-yl}2-methylpropane-2-sulfinamide (from Step E) was added 5 mL anhydrous 4 M HCl in dioxane and the solution stirred for 15 min at room temperature. The reaction was concentrated to dryness and azeotroped with toluene (2×5 mL) to remove excess HCl. The residue was then taken up in anhydrous dichloromethane, set under nitrogen atmosphere, cooled to 0° C. with ice/water bath and then benzyl chloroformate (0.32 mL, 2.22 mmol) was slowly added via syringe followed by diisopropylethyl amine (1.19 mL, 6.66 mmol) and the resulting solution stirred for 2 h at 0° C. The solution was concentrated to dryness under vacuum and the residue was purified via preparative plates (4×1000 μM) eluding with 20% ethyl acetate in hexane to afford the title compound (703 mg, 71%). m/z (ES) 446, 448 (M, M+2)⁴, 468, 470 (MNa, MNa+2)⁺. ¹HNMR (500 MHz, CDCl₃) δ: 7.32 (s, 1H), 7.30 (br d, J=7.5, 1H), 7.27-7.25 (m, 2H), 7.21-7.18 (m, 1H), 5.92 (ddd, J=7.1, 10.3, 17.4 Hz, 1H), 5.23 (d, J=10.4 Hz, 1H), 5.18 (d, J=17.4 Hz, 1H), 4.75 (d, J=4.2 Hz, 1H), 3.88-3.82 (m, 1H), 3.33 (d, J=9.4 Hz, 1H), 1.19 (s, 9H), 0.94 (s, 91-1), 0.09 (s, 3H), −0.14 (s, 3H).

Intermediates related to those described above of varying stereoechemistry may be prepared from the appropriate starting materials using the procedure described above.

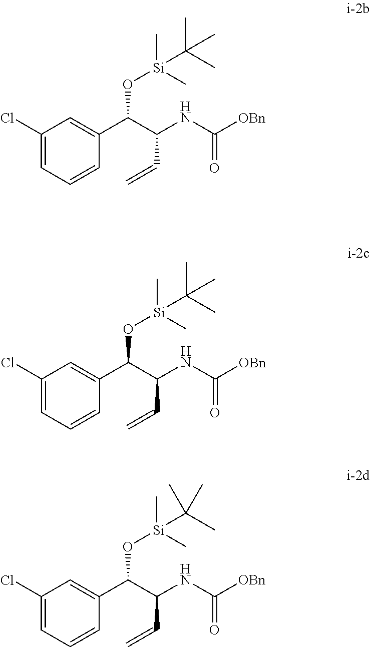

### Intermediate 3

Tert-butyl(5R)-2-(4-aminobenzyl)-5-[(R)-{[tert-butyl(dimethyl)silyl]oxy}(phenyl)methyl]pyrrolidine-1-carboxylate (i-3)

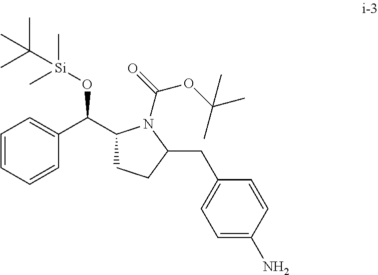

Step A: Benzyl{4-[(3E, 5R, 6R)-5-{[(benzyloxy)carbonyl]amino-6-{[tert-butyl (dimethyl)silyl]oxy}-6-(3-chlorophenyl)-2-oxohex-3-en-1-yl]phenyl}carbamate

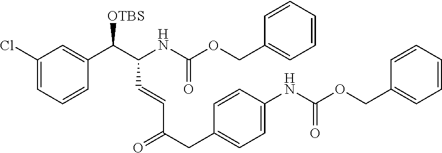

To a solution of benzyl [3-(2-oxobut-3-en-1-yl)phenyl] carbamate (i-1) (820 mg, 2.80 mmol) and ((1R)-1-[(R)-[tert-butyl(dimethyl)silyl]oxy)(3-chlorophenyl)methyl]prop-2-en-1-yl)carbamate (i-2) (500 mg; 1.12 mmol) in 7 mL of anhydrous dichloromethane was added the Zhan I catalyst (740 mg, 1.12 mmol) and the resulting green solution was heated to 40° C. overnight under nitrogen atmosphere. The reaction was concentrated to dryness and the residue purified via preparative plates (4×1000 μM) eluting with 40% ethyl acetate in hexane to afford the title compound (348 mg, 50%). m/z (ES) 713, 715 (M, M+2)⁺, 735, 737 (MNa, MNa+2)⁺.

Step B: 4-({(5R)-5-[(R)-([tert-butyl(dimethyl)silyl]oxy}(phenyl)methyl]pyrrolidin-2-yl)methyl)aniline

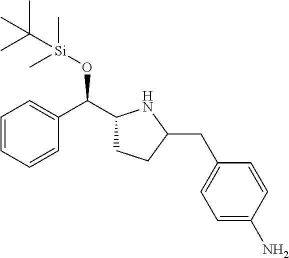

To a solution of 328 mg (0.46 mine!) of benzyl{4-[(3E, 5R, 6R)-5-{[(benzyloxy)carbonyl]amino-6-{[tert-butyl (dimethyl)silyl]oxy}-6-(3-chlorophenyl)-2-oxohex-3-en-1-yl]phenyl}carbamate (from Step A) in 25 mL ethanol was added 10% palladium on carbon and the suspension was set under hydrogen atmosphere via a balloon of hydrogen gas. The reaction was stirred under hydrogen for 1 h at room temperature. TLC proved that the reaction was complete. The catalyst was filtered off using a Oilmen 0.45 μM PTFE syringe filter and washed with ethanol (4×5 mL). The filtrate was concentrated to dryness under vacuum and the residue purified by preparative plate (3 x 1000 μM) eluding with 5% methanol in dichloromethane to afford the title compound (121 mg, 66%). m/z (ES) 397 (MH)⁺.

Step C: Tert-butyl(5R)-2-(4-aminobenzyl)-5-[(R)-{[tert-butyl(dimethyl)silyl]oxy}(phenly)methyl]pyrrolidine-1-carboxylate (i-3)

To a solution of 121 mg (0.315 mmol) of 4-({(5R)-54(R)-([tert-butyl(dimethyl)silyl]oxyl}(phenyl)methylipyrrolidin-2-yl}methyl)aniline in 5 mL of anhydrous THF (from Step B) was added tert-butyl carbonate (69 mg, 0.315 mmol), followed by TEA (44 μL, 0.315 mmol) and the resulting solution stirred at room temperature under nitrogen atmosphere overnight. The reaction mixture was put directly on a preparative plate (1500 μM) and eluted with 30% ethyl acetate in hexane to afford the title compound (100 mg, 64%). m/z

(ES) 497 (MH)⁺, 397 (M-Boc)⁺. ¹HNMR (500 MHz, CDCl₃) δ: 7.40-7.30 (m, 514), 6.75-6.68 (m, 2H), 6.56-6.51 (m, 2H), 5.52-5.48 (m, 1H), 5.32-5.28 (m, 1H), 4.16-4.06 (m, 2H), 3.88-3.82 (m, 1H), 3.76-3.70 (m, 1H), 3.55-3.48 (m,2H), 2.74 (br d, J=11.8 Hz, 1H), 2.44 (br d, J=11.8 Hz, 1H), 2.05-1.94 (m, 1H), 1.90-1.82 (m, 1H), 1.60 (s, 9H), 1.50-1.42 (m, 1H), 1.32-1.22 (m, 2H), 1.10-1.02 (m,1H), 0.95 (s, 91-1), 0.08 (s, 3H), -0.15 (s, 3H).

### Separation of Intermediate 4A and Intermediate 4B

Tert-butyl (2S, 5R)-2-(4-aminobenzyl)-5-[(R)-{[tert-butyl(dimethyl)silyl]oxy}(phenyl)methyl]pyrrolidine-1-carboxylate (i-4a);
Tert-butyl (2R, 5R)-2-(4-aminobenzyl)-5-[(R)-{[tert-butyl(dimethyl)silyl]oxy}(phenyl)methyl]pyrrolidine-1-carboxylate (i-4b)

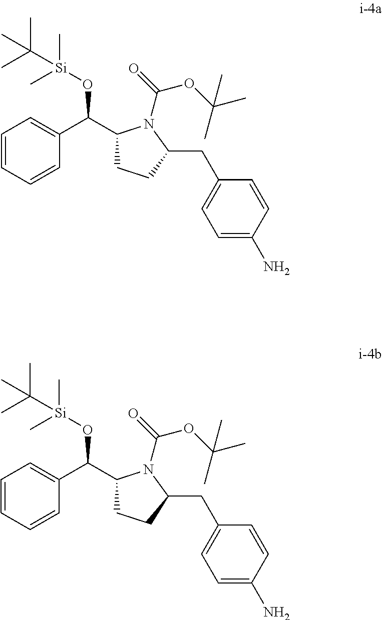

Step A: Tert-butyl (2S, 5R)-2-(4-aminobenzyl)-5[(R)-{[tert-butyl(dimethyl)silyl]oxy}(phenyl)methyl]pyrrolidine-1-carboxylate (i-4a) and tert-butyl (2R, 5R)-2-(4-aminobenzyl)-5-[(R)-{[tert-butyl(dimethyl)silyl]oxy}(phenypmethyl]pyrrolidine-1-carboxylate (i-4b)

The intermediate i-3 (tert-butyl(5R)-2-(4-aminobenzyl)-5-[(R)-{[tert-butyl(dimethyl)silyl]oxy}(phenyl)methyl]pyrrolidine-1-carboxylate (4:1 mixture of cis and trans) was taken up in methanol and purified via the Berger Multigram SFC (supercritical) using an eluent of 30% methano1:60% carbon dioxide to separate the two diastereomers. The first isomer of the column was labeled minor isomer 1 and the second isomer was labeled major isomer 2.

i-4a: m/z (ES) 497 (MH)⁺, 397 (M-Boc)⁺. ¹HNMR (500 MHz, CDCl₃) δ: 7.40-7.30 (m, 5H), 6.75-6.68 (m, 2H), 6.56-6.51 (m, 2H), 5.52-5.48 (m, 1H), 5.32-5.28 (m, 1H), 4.16-4.06 (m, 2H), 3.88-3.82 (m, 1H), 3.76-3.70 (m, 1H), 3.55-3.48 (m,2H), 2.74 (br d, J=11.8 Hz, 1H), 2.44 (br d, J−11.8 Hz, 1H), 2.05-1.94 (m, 1H), 1.90-1.82 (m, 1H), 1.60 (s, 9H), 1.50-1.42 (m, 1H), 1.32-1.22 (m, 2H), 1.10-1.02 (m,1H), 0.95 (s, 9H), 0.92 (d, J=11.8 Hz, 1H), 0.12 (br d, J=14.0 Hz, 3H), −0.04 (s, 3H). Eluted 8.70 min on SFC, isomer 2
i-4b: m/z (ES) 497 (MH)⁺, 397 (M-Boc). ¹HNMR (500 MHz, CDCl₃) δ: 7.40-7.30 (m, 5H), 6.76-6.68 (m, 2H), 6.56-6.51 (m, 2H), 5.52-5.48 (m, 1H), 5.32-5.28 (m, 1H), 4.16-4.06 (m, 2H), 3.88-3.82 (m, 1H), 3.76-3.70 (m, 1H), 3.60-3.46 (m,2H), 2.72 (br d, J=12.0 Hz, 1H), 2.44 (br d, J=12.2 Hz, 1H), 2.05-1.94 (m, 1H), 1.90-1.82 (m, 1H), 1.64 (s, 9H), 1.50-1.42 (m, 1H), 1.32-1.22 (m, 2H), 1.10-1.02 (m,1H), 0.95 (s, 9H), 0.14 (br d, J−13.8 Hz, 3H), 0.09 (s, 3H). Eluted 7.78 min on SFC, isomer 1.

### Synthesis of Intermediate 4A and Intermediate 4B

Tert-butyl (2S, 5R)-2-4-aminobeenzyl)-5-[(R)-{[tert-butyl(dimethyl)silyl]oxy}(phenyl)methyl]pyrrolidine-1-carboxylate (i-4a);

Tert-butyl (2R, 5R)-2-4-aminobenzyl)-5-[(R)-{[tert-butyl(dimenthyl)silyl]oxy}(phenyl)methyl]pyrrolidine-1-carboxylate (i-4b)

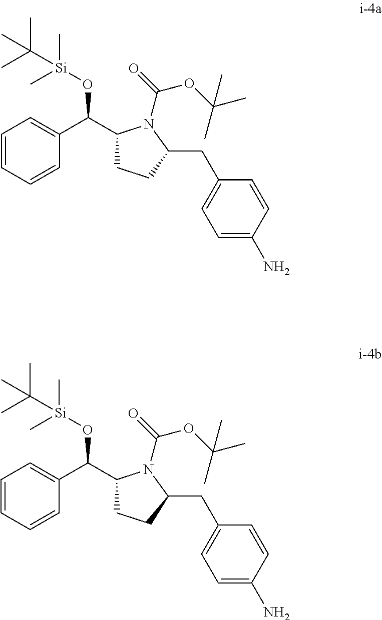

Step A: (45)-3-Hex-5-ynoyl-4-phenyl-1,3-oxazolidin-2-one

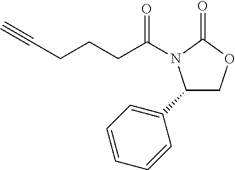

To a solution of 69.0 g (615 mmol) of 5-hexynoic acid and 214 mL (1540 mmol) of triethylamine in 1.0 L of anhydrous tetrahydrofuran at −25° C. under an atmosphere of nitrogen was added 83.0 mL (677 mmol) of trimethylacetyl chloride over 20 min. Upon addition a white precipitate formed and the resulting suspension was stirred for 2 h. Next, 28.7 g (677 mmol) of anhydrous lithium chloride and 100.0 g (615.0 mmol) of (4S)-4-phenyl-1,3-oxazolidin-2-one were added sequentially and the mixture was allowed to gradually warm to ambient temperature over 12 h. All volatiles were removed in vacua and the residue was diluted with water (1 L) and extracted with ethyl acetate (3×300 mL). The combined organic layers were washed with brine (250 mL), dried over magnesium sulfate, filtered and concentrated in vacua. The crude residue was purified by silica gel chromatography eluting with a 5-50% ethyl acetate in hexanes gradient to afford the title compound as a colorless solid (135 g, 85.4%). ¹H NMR (500 MHz, CDCl₃): δ 7.40-7.37 (m, 2H), 7.36-7.32 (m, 1H), 7.31-7.28 (m, 2H), 5.42 (dd, J=8.9, 3.7 Hz, 1H), 4.69 (t, J=8.9 Hz, 1H), 4.28 (dd, J=9.2, 3.7 Hz, 1H), 3.13-3.02 (m, 2H), 2.24-2.21 (m, 2H), 1.94 (t,J=2.6 Hz, 1H), 1.84 (quintet, J=7.1 Hz, 2H). LC-MS: m/z (ES) 258.2 (MH)⁺.

Step B: (4S)-3-{(2R)-2-[(S)-Hydroxy(phenyl)methyl]hex-5-ynoyl}-4-phenyl-1,3-oxazolidin-2-one

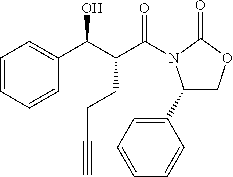

To a stirred solution of 56.8 g (221 mmol) of (4S)-3-hex-5-ynoyl-4-phenyl-1,3-oxazolidin-2-one from step A above in 265 mL of anhydrous ethyl acetate at ambient temperature under an atmosphere of nitrogen was added 6.31 g (66.2 mmol) of anhydrous magnesium chloride, 61.5 mL (442 mmol) of triethylamine, 26.9 mL (265 mmol) of benzaldehyde and 42.3 mL (331 mmol) of chlorotrimethylsilane and the resulting mixture was stirred for 72 h. The heterogeneous reaction mixture was filtered through a 300 mL plug of silica gel eluting with an additional 1L of ethyl acetate. The filtrate was evaporated to dryness in vacua and the residue suspended in 265 mL of methanol and 10 mL of trifluoroacetic acid. The resulting mixture was stirred at ambient temperature under nitrogen for 5 h during which time the reaction became homogeneous. All volatiles were then removed in vacuo and the residue was purified by silica gel chromatography eluting with a 5-15% ethyl acetate in hexanes gradient to afford the title compound as a white solid (65.0 g, 81.2%). ¹H NMR (500 MHz, CDCl₃): δ 7.30-7.28 (m, 8H), 7.09-7.07 (m, 2H), 5.42 (dd, J=8.7, 3.7 Hz, 1H), 4.76-4.72 (m, 1H), 4.72-4.67 (m, 1H), 4,65 (t, J=8.7 Hz, 1H), 4.18 (dd, J=8.7, 3.7 Hz, 1H), 3.05 (d, J=7.8 Hz, 1H), 2.24 (td, J=7.1, 2.5 Hz, 2H), 2.00-1.93 (m, 2H), 1.67-1.61 (m, 1H). LC-MS: m/z (ES) 346.1 (MH−H₂O)⁺, 386.0 (MNa)⁺.

Step C: (2R)-2-[(S)-Hydroxy(phenyl)methyl]hex-5-ynoic acid

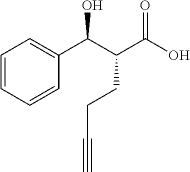

To a stirred solution of 65.0 g (179 mmol) of (4S)-3-{(2R)-2-[(S)-hydroxy(phenyl)methyl]hex-5-ynoyl}-4-phenyl-1,3-oxazolidin-2-one from Step B above in 1050 mL of a 20 to 1 mixture of anhydrous tetrahydrofuran to water at 0° C. under an atmosphere of nitrogen was added 77.0 mL (894 mmol) of a 35% aqueous hydrogen peroxide solution at a rate slow enough to keep the internal temperature below 3° C. Next, 395 mL (395 mmol) of a 1.0 M aqueous lithium hydroxide solution was added at a rate slow enough to keep the internal temperature of the reaction below 5° C. and the resulting mixture was stirred for 3 h at 0° C. The reaction was quenched with 755 mL (984 mmol) of a 1.3 M aqueous sodium sulfite solution at a rate slow enough to keep the internal temperature of the mixture below 5° C. All volatiles were removed in vacuo and the remaining aqueous phase was extracted with ethyl acetate (3×200 mL). The aqueous phase was then cooled to 0° C. and acidified with a 6 M aqueous hydrogen chloride solution until a pH of 3 was achieved. The aqueous phase was then extracted with ethyl acetate (3×300 mL) and the combined organics were washed with brine (100 ml), dried over magnesium sulfate, filtered and evaporated in vacuo. The residue was purified by silica gel chromatography eluting with a 5-10% ethyl acetate and 3% acetic acid in hexanes gradient to afford the title compound as a colorless gum (32.0 g, 82.0%). ¹H NMR (500 MHz, CDCl₃): δ 7.39-7.28 (m, 5H), 4.85 (d, J=8.2, 1H), 3.03-2.97 (m, 1H), 2.29-2.15 (m, 2H), 1.97 (t, J=2.5 Hz, 1H), 1.93-1.82 (m, 1H), 1.62-1.55 (m, 1H). LC-MS: m/z (ES) 201.0 (MH−H₂O)⁺.

Step D: 2R -2-[(S)-{[Tert-butyl(dimethyl)silyl]oxy}(phenyl)methyl]hex-5-ynoic acid

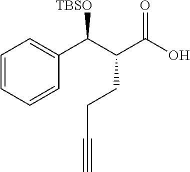

To a stirred solution of 32.0 g (147 mmol) of (2R)-2-[(S)-hydroxy(phenyl)methyl]hex-5-ynoic acid from Step C above in 500 mL of anhydrous acetonitrile at ambient temperature under an atmosphere of nitrogen was added 77.0 mL (513 mmol) of 1,8-diazabicyclo[5.4.0]undec-7-ene 22 mL followed by 66.3 g (440 mmol) of tert-butyldimethylsilyl chloride in three portions over 10 min. The reaction mixture was stirred for 4 h then evaporated in vacuo to remove all volatiles. The residue was diluted with 300 mL of dichloromethane and 100 mL of water. A 1.0 M aqueous hydrogen chloride solution was added to the mixture until a pH of 3 was achieved in the aqueous layer. The phases were separated and the aqueous phase was extracted with dichloromethane (2×100 mL). The combined organics were washed with water (50 mL), brine (50 mL) then dried over magnesium sulfate. After filtration and evaporation in vacuo the residue was dissolved in 350 mL of methanol and 350 mL (280 mmol) of a 0.8 M aqueous potassium carbonate solution was added. The resulting mixture was stirred for 1.5 h then evaporated in vacuo to remove all volatiles. The residue was diluted with 300 mL of dichloromethane and the aqueous phase was acidified with a 5.0 M aqueous hydrogen chloride solution until a pH of 3 was achieved. The phases were separated and the aqueous phase was extracted with dichloromethane (2×100 mL). The combined organics were washed with water (50 mL), brine (50 mL) then dried over magnesium sulfate, filtered and evaporated in vacuo. The residue was purified by silica gel chromatography eluting with a 3-15% ethyl acetate in hexanes gradient to afford the title compound as a colorless solid (42.3 g, 86.6%). ¹HNMR (500 MHz, CDCl₃): δ 7.36-7.27 (m, 5H), 4.78 (d, J=8.7, 1H), 2.90-2.86 (m, 1H), 2.19-2.11 (m, 1H), 2.10-2.03 (m, 1H), 1.90 (t, J=2.6 Hz, 1H), 1.75-1.67 (m, 1H), 1.41-1.34 (m, 1H), 0.83 (s, 9H), 0.02 (s, 3H), -0.27 (s, 3H). LC-MS: m/z (ES) 333.2 (MH)⁺.

Step E: 4-Methoxybenzyl {(1R)-1-[(R)-{[tert-butyl(dimethyl)silyl]oxy}(phenyl)methyl]pent-4-yn-1-yl}carbamate

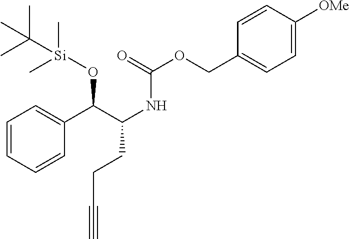

To a solution of 40.0 g (120 mmol) of (2R)-2-[(S)-{[tert-butyl(dimethyl)silyl]oxy}(phenyl)methyl]hex-5-ynoic acid from Step D above and 33.5 mL (241 mmol) of triethylamine in 400 mL of anhydrous toluene at ambient temperature under an atmosphere of nitrogen was added 37.5 mL (132 mmol) of diphenylphosphoryl azide. The mixture was stirred for 5 h and then 37.5 mL (301 mmol) of 4-methoxybenzyl alcohol was added. The resulting mixture was heated to 105° C. for 16 h, cooled to ambient temperature and then diluted with 250 mL of a saturated aqueous bicarbonate solution. The phases were separated and the aqueous phase was extracted with ethyl acetate (2×150 mL). The combined organics were washed with water (100 mL), brine (100 mL) then dried over magnesium sulfate, filtered and evaporated in vacuo. The crude residue was purified by silica gel chromatography eluting with 3-10% ethyl acetate in hexanes to afford the title compound as a colorless oil (50.9 g, 90.5%). ¹H NMR (500 MHz, CDCl₃): 7.28-7.21 (m, 7H), 6.87 (d, J=8.4 Hz, 2H), 4.92 (s, 2H), 4.77-4.59 (m, 2H), 3.89-3.84 (m, 1H), 3.81 (s, 3H), 2.30-2.22 (m, 2H), 1.95 (m, 1H), 1.91-1.85 (m, 1H), 1.57-1.50 (m, 1H), 0.89 (s, 9H), 0.06 (s, 3H), -0.15 (s, 3H). LC-MS: m/z (ES) 468.1 (MH)⁺, 490.0 (MNa)⁺.

Step F: 4-methoxybenzyl [(1R)-1-[(R)-{[tert-butly(dimethyl)silyl]oxy}(pheynyl)mehtyl]-5-(4-nitrophenyl)pent-4-yn-1-yl]carbamate

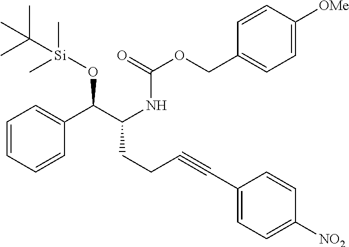

To a solution of acetylene (from Step E, 40g, 80 mmol) and 4-iodonitrobenzene (21.8 g, 88 mmol) in anhydrous DMF (500 ml) was added triethylamine (111 mL, 797 mmol). Pd(dppf)Cl₂ (1.95 g, 239 mmol) and copper(I) iodide (910 mg, 4.78 mmol) was added and the mixture degassed with nitrogen (bubble 15 min) and the resulting solution stirred at room temperature for 5 h. The mixture was poured into water (1200 m) and extracted with EtOAc (3×300 mL). The combined organics were then washed with water (2×500 mL), sat. NaCl (200 mL), dried over magnesium sulfate, filtered and evaporated under vacuum. Residue was purified by MPLC (Horizon Biotage 2× Flash 65i) eluting with a gradient of 0-30% ethyl acetate in hexane to give 41 g (84%) as a dark red oil. %). ¹HNMR (500 MHz, CDCl₃): 8.11-8.04 (m, 2H), 7.94-8.01 (m, 1H), 7.38-7.21 (m, 8H), 6.87 (d, J=8.4 Hz, 2H), 4.98 (s, 2H), 4.77-4.59 (m, 2H), 4.00-3.95 (m, 3H), 3.81 (s, 3H), 2.56 (t, J=7.1 Hz, H=2H), 2.00-1.95 (m, 1H), 1.66-1.61 (m, 1H), 0.93 (s, 9H), 0.10 (s, 3H), -0.10 (s, 3H). LC-MS: m/z (ES) 589.3 (MH)⁺, 611.2 (MNa)⁺.

Step G: 4-methoxybenzyl [(1R)-1-[(R)-{[tert-butyl(dimethypsilyl]oxy}(phenyl)methyl]-5-(4-nitrophenyl)-4-oxopentyl]carbamate

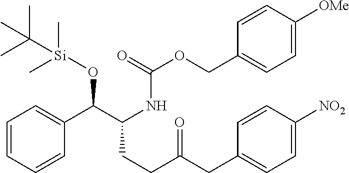

To a solution of nitrophenyl acetylene (from Step F, 41 g, 65.5 mmol) in DMF (40 ml) was added pyrrolidine (14 mL, 196.5 mmol) and the resulting mixture heated at 80° C. for 3 h. The mixture was cooled to room temperature and a 10% solution of acetic acid in water (110 ml) was added, and the resulting solution stirred at room temperature for another 3 h. The mixture was poured into water (300 ml) and extracted with EtOAc (3×250 ml); combined EtOAc layers were washed with water (2×250 ml), sat. NaCl (100 ml), dried over MgSO₄, filtered and evaporated. The residue was purified by Horizon Flash 75 eluting with a gradient rising from 100% Hexanes to 50% EtOAc in Hexanes to give 34 g (81%) as a dark orange oil. ¹H NMR (500 MHz, CDCl₃): 8.17-8.14 (m, 2H), 7.32-7.23 (m, 9H), 6.87 (d, J=8.4 Hz, 2H), 4.96 (d, J=12.2 Hz, 1H), 4.90 (d, J=12.1 Hz, 1H), 4.72 (d, J=3 Hz, 1H), 4.16-4.13 (m, 1H), 3.81 (s, 3H), 3.71-3.77 (m, 2H), 2.65-2.52 (m, 21-1), 1.97-1.92 (m, 1H), 1.72-1.60 (m, 1H), 0.93 (s, 9H), 0.05 (s, 3H), -0.13 (s, 3H).

Step H: (2R, 5S)-2-[(R)-{[tert-butyl(dimethyl)silyl]oxy}(phenyl)methyl]-5-(4-nitrobenzyl)pyrrolidine(2R, 5R)-2-{[tert-butyl(dimethyl)silyl]oxy }(phenyl)methyl1-5-(4-nitrobenzyl)pyrrolidine

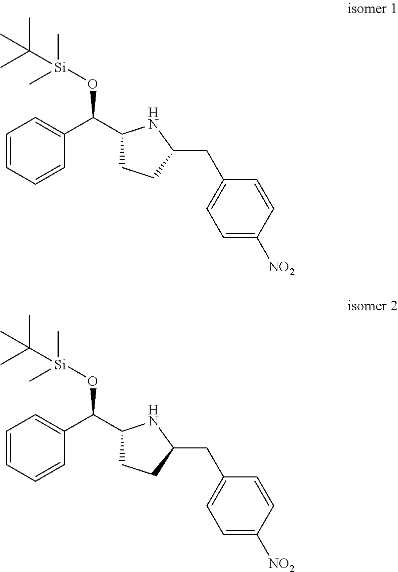

To a solution of MOZ protected ketone amine (from Step G, 34 g, 56 mmol) in DCM (350 ml) was added TFA (256 ml) and the resulting mixture stirred at room temperature for 1.5 h. The solution was evaporated under vacuum and residue partitioned between DCMand-sat. NaHCO₃. The organic layer was dried over MgSO₄, filtered and evaported. The residue was dissolved in MeOH (750 ml) and cooled to 0° C. via ice/water bath. Sodium cyanoborohydride (21.2 g, 337 mmol) was then added and the resulting mixture was stirred overnight to allow to warm to room temperature. The mixture was quenched by addition of water and the organics removed under vacuum. The aqueous layer was then extracted with EtOAc (×2) and the combined EtOAc layers washed with sat. NaCl, dried over MgSO₄, filtered and evaporated under vacuum. The residue was purified by column chromatography on silica (eluent: gradient rising from 100% Hexanes to 35% EtOAc in Hexanes) to give 16.4 g (63.4%) of the first isomer, (2R, 5S)-2-[(R)-{[tert-butyl(dimethyl) silyl]oxy}(phenyl)methyl]-5-(4-nitrobenzyl)pyrrolidine, and 3.1 g (12%) of the second isomer (2R, 5R)-2-[(R)-{[tert-butyl(dimethyl)silyl]oxy} (phenyl)methyl]-5-(4-nitrobenzyl)pyrrolidine.

### Isomer 1: LC-MS: m/z (ES) 427.3 (MH)⁺

### Isomer 2: LC-MS: m/z (ES) 427.3 (MH)⁺

Step I: Tert-butyl (2R, 5S)-2-[(R)-{[tert-butyl(dimethyl)silyl]oxy}(phenyl)methyl]-5-(4-nitrobenzyl)pyrrolidine-1-carboxylate

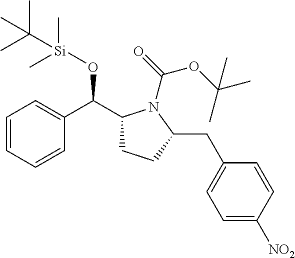

To a solution of tert-butyl (2R, 5S)-2-[(R)-{[tert-butyl(dimethyl)silyl]oxy}(phenyl)mehtyl]-5-(4-nitrobenzyl)pyrrolidine-1-carboxylate (12 g, 42.5 mmol) in anhydrous THF was added Boc anhydride (9.3 g, 42.5 mmol) followed by TEA (17.76 mL, 127.4 mmol) and the resulting solution stirred at room temperature under nitrogen atmosphere for 2 h. The mixture was washed with water (100 mL) and extracted with ethyl acetate (2×200 mL). The organics were dried over sodium sulfate, filtered, and concentrated under vacuum. The residue was purified via Horizon Biotage MPLC (65i silica gel column) eluting with a gradient of 20-75% ethyl acetate in hexane to afford the desired product. LC-MS: m/z (ES) 527.3 (MH)⁺, 549.2 (MNa)⁺.

Step J: Tert-butyl (2R, 5R)-2-[(R)-{[tert-butyl(dimethyl)silyl]oxy}(phenyl)methyl]-5-(4-nitrobenzyl)pyrrolidine-1-carboxylate

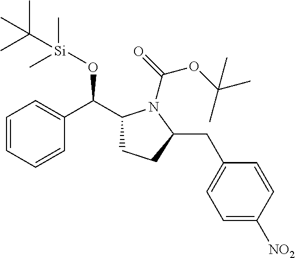

Prepared in the same manner as Step I but replacing the cis pyrrolidine isomer with the trans isomer, (2R, 5R)-2-[(R)-{[tert-butyl(dimethypsilyl]oxy}(phenyl)methyl]-5-(nitrobenzyl)pyrrolidine. LC-MS: m/z (ES) 527.3 (MH)⁺, 549.2 (MNa)⁺.

Step K: Tert-butyl (2S, 5R)-2-(4-aminobenzyl)-5-[(R)-{[tert-butyl(dimethyl)sily]oxy}(phenypmethyl]pynolidine-1-carboxylate (i-4a);

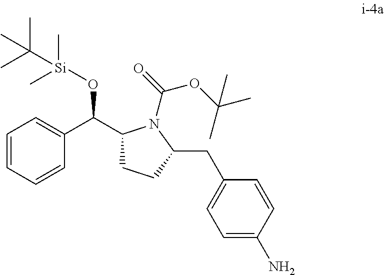

A 500 mL parr shaker flask was charged with 10% Pd/c (4.75 g) and to this was added 100 mL of methanol to cover the catalyst. A solution of the nitro intermediate from Step I (8.5 g, 18.5 mmol) in methanol (80 mL) was then added to the suspension, followed by 15.4 mL of 1.0 M hydrogen chloride in methanol solution. The reaction vessel was set under 50 PSI hydrogen gas and the mixture aggitated overnight. An aliquot was taken and analyzed through the LC-MS which showed complete reaction.

The catalyst was filtered off using celite and washed with methanol (2×100 mL). The filtrate was concentrated to dryness and the product was purified via Horizon MPLC (65i silica column) eluting with a gradient rising from 0% to 30% ethyl acetate in hexane to afford the title compound (6.2g, 72%). m/z (ES) 497 (MH)⁺, 397 (M-Boc)⁺. ¹HNMR (500 MHz, CDCl₃) δ: 7.38-7.29 (m, 5H), 6.76-6.68 (m, 2H), 6.55-6.50 (m, 2H), 5.52-5.49 (m, 1H), 5.30-5.27 (m, 1H), 4.15-4.05 (m, 2H), 3.86-3.81 (m, 1H), 3.76-3.71 (m, 1H), 3.55-3.47 (m,2H), 2.74 (br d, J=11.7 Hz, 1H), 2.44 (br d, J=11.7 Hz, 1H), 2.05-1.93 (m, 1H), 1.90-1.83 (m, 1H), 1.60 (s, 9H), 1.50-1.42 (m, 1H), 1.31-1.21 (m, 2H), 1.10-1.02 (m,1H), 0.95 (s, 9H), 0.92 (d, J=11.8 Hz, 1H), 0.13 (br d, J=14.0 Hz, 3H), −0.05 (s, 3H)

Step L: Tert-butyl (2R, 5R)-2-(4-aminobenzyl)-5-[(R)-{[tert-butyl(dimethyl)sily]oxy}(phenypmethyl]pyrrolidine-1-carboxylate (i-4b)

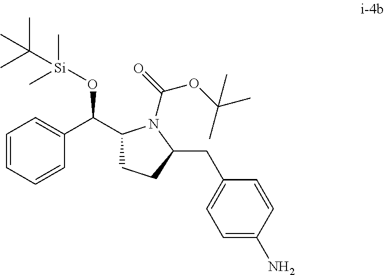

Prepared in the same manner as Step K but replacing the cis pyrrolidine isomer with the trans isomer, Tert-butyl (2R, 5R)-2-[(R)-{[tert-butyl(dimethy)silyl]oxy}(phenyl)methyl]-5-(4-nitrobenzyl)pyrrolicline-1-carboxylate, m/z (ES) 497 (MH)⁺, 397 (M-Boc)⁺. ¹HNMR (500 MHz, CDCl₃) δ: 7.41-7.30 (m, 5H), 6.73-6.67 (m, 2H), 6.56-6.50 (m, 2H), 5.52-5.48 (m, 1H), 5.33-5.28 (m, 1H), 4.15-4.06 (m, 2H), 3.86-3.81 (m, 1H), 3.76-3.70 (m, 1H), 3.59-3.46 (m,2H), 2.72 (br d, J=12.0 Hz, 1H), 2.44 (br d, J=12.0 Hz, 1H), 2.05-1.93 (m, 1H), 1.90-1.82 (m, 1H), 1.64 (s, 9H), 1.49-1.42 (m, 1H), 1.32-1.20 (m, 2H), 1.10-1.02 (m,1H), 0.95 (s, 9H), 0.14 (br d, J=13.7 Hz, 3H), 0.10 (s, 3H).

The following intermediates were prepared from the appropriate starting materials using the procedures described above for intermediate i-4a.

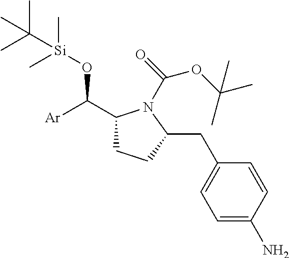

| Intermediate | Ar | Calc. Mass | MS (e/z) (MH)⁺ |
|---|---|---|---|
| i-4c | 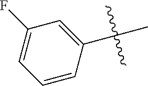 | 514.30 | 515.30 |
| i-4d | 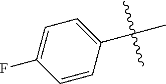 | 514.30 | 515.30 |
| i-4e | 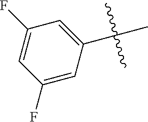 | 532.30 | 533.30 |
| i-4f | 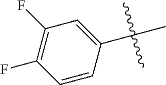 | 532.30 | 533.30 |

The following intermediates were prepared from the appropriate starting materials using the procedures described above for intermediate i-4b.

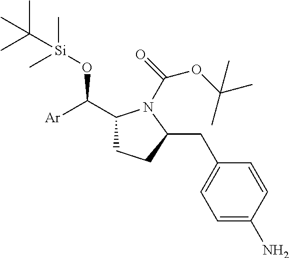

| Intermediate | Ar | Calc. Mass | MS (e/z) (MH)⁺ |
|---|---|---|---|
| i-4g | 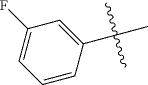 | 514.30 | 515.30 |
| i-4h | 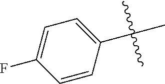 | 514.30 | 515.30 |

### Intermediate 5

Tert-butyl(5R)-2-(4-aminobenzyl)-5-[(R)-{[tert-butyl(dimethyl)sily]oxy}(3-chlorophenyl)methyl]pynolidine-1-carboxylate (i-5)

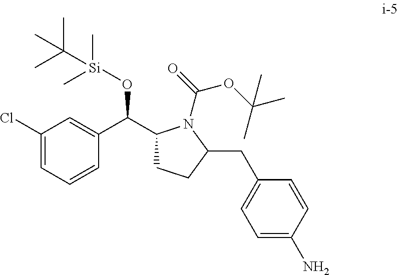

Step A: 4-({(5R)-5-[(R)-([tert-butyl(dimethyl)silyl]oxy}(3-chlorophenyl)methyl]pyrrolidin-2-yl}methyl)aniline

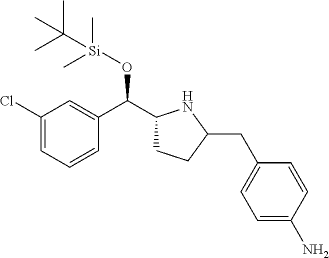

To a solution of 100 mg (0.15 mmol) of benzyl {4-[(3E, 5R, 6R)-5-{[(benzyloxy)carbonyl]amino-6-{[tert-butyl (dimethyl)silyl]oxy}-6-(3-chlorophenyl)-2-oxohex-3-en-1-yl]phenyl}carbamate (from Step A, i-3) in 8 mL ethyl acetate was added 10% palladium on carbon and the suspension was set under hydrogen atmosphere via a balloon of hydrogen gas. The reaction was stirred under hydrogen for 8 h at room temperature. The catalyst was filtered off using a Gilmen 0.45 uM PTFE syringe filter and washed with ethyl acetate (4×2 mL). The filtrate was concentrated to dryness under vacuum and the residue purified by preparative plate (1000 μM) eluding with 5% methanol in dichloromethane to afford the title compound (33 mg, 51%). m/z (ES) 430, 432 (M, M+2)⁺.

Step B: Tert-butyl(5R)-2-(4-aminobenzyl)-5-[(R)-{[tert-butyl(dimethyl)silyl]oxy}(3-chlorophenyl)methyl]pyrrolidine-carboxylate (i-5)

To a solution of 33 mg (0.07 mmol) of 4-({(5R)-5-[(R)-([tert-butyl(dimethyl)silyl]oxy}(3-chlorophenyl)methyl]pyrrolidin-2-yl}methypaniline in 1 mL of anhydrous THF (from Step A) was added tert-butyl carbonate (15.3 mg, 0.07 mmol), followed by TEA (13 uL, 0.07 mmol) and the resulting solution stirred at room temperature under nitrogen atmosphere overnight. The reaction mixture was put directly on a preparative plate (500 uM) and eluted with 30% ethyl acetate in hexane to afford the title compound (25 mg, 78%). m/z (ES) 530, 532 (M, M+2)⁺, 430, 432 (M-Boc, M-Boc+2)⁺.

### Intermediate 6

4-{4-[4-(Trifluoromethyl)phenyl]-1,3-thiazol-2-yl}benzenesulfonyl chloride (i-6)

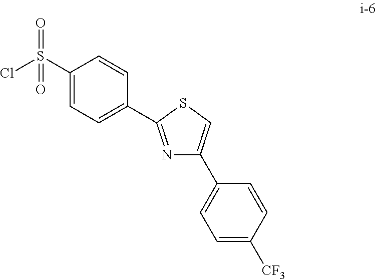

Intermediate 6 can be prepared according to published procedures, for example, Ikemoto et al., Tetrahedron 2003, 59, 1317-1325.

### Intermediate 7

2-Methyl-5,6-dihydro-4H-cyclopenta [α] [1,3] thiazole-4-carboxylic acid (i-7)

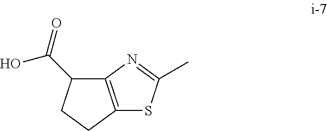

Step A: Ethyl 2-methyl-5,6-dihydro-4H-cyclopenta [α] [1,3] thiazole-4-carboxylate

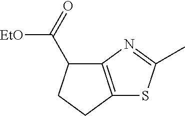

To a solution of ethyl 2-oxocyclopentane-2-carboxylate (56 g, 359 mmol) in chloroform (500 mL) cooled at 0° C. was added bromine (18.5 mL, 359 mmol) over ˜20 min. After complete addition mixture allowed to warm to room temperature and stirred overnight. Nitrogen gas bubbled through mixture for 90 min to remove most of HBr. Washed with water (500 mL), sat. NaHCO₃ (250 mL), sat. NaCl (200 mL), dried over MgSO₄, filtered and evaporated. Residue dissolved in EtOH (500 mL) and thioacetamide (26.9 g, 359 mmol) added, mixture stirred at room temperature for 1 h then at reflux overnight. The mixture was cooled and evaporated, and the residue partitioned between DCM and sat. NaHCO₃, organic layer washed with sat. NaCl, dried over MgSO₄, filtered and evaporated. The Residue purified by MPLC (Biotage Horizon: 2× FLASH 65i) eluent: 100% Hexanes (450 mL), gradient rising from 100% Hexanes to 25% EtOAc in Hexanes (1400 mL), then 25% EtOAc in Hexanes to give the title compound (32 g, 42%) as a dark oil. ¹HNMR (500 MHz, CDCl₃) δ: 4.22 (q, J=7.0 Hz, 2H), 3.96 (m, 1H), 3.04 (m, 1H), 2.88 (m, 1H), 2.76 (m, 2H), 2.70 (s, 3H), 1.30 (t, J=7.0Hz, 3H).

Step B: 2-Methyl-5,6-dihydro-4H-cyclopenta [α] [1,3] thiazole-4-carboxylic acid (i-7)

To a solution of 31.5 g (149 mmol) of ethyl 2-methyl-5,6-dihydro-4H-cyclopenta [α] [1,3] thiazole-4-carboxylate in THF (450 mL) and methanol (100 mL) (from step A) was added a solution of lithium hydroxide (149 mL of a 1M solution, 149 mmol) and the resulting mixture stirred at room temperature for 3 h. Organics removed by evaporation and aqueous residue extracted with Et₂O (2×250 mL) and acidified to pH 3 by the addition of 1 M HCl (˜170 mL) and saturated with solid NaCl. Extracted with DCM (3×250 mL), combined DCM layers dried over MgSO₄, filtered and evaporated. Extracted with DCM (3×250 mL), combined DCM layers dried over MgSO₄, filtered and evaporated. Residue triturated with acetonitrile, filtered and dried to give the title compound (7.1 g, 26%) as an off white solid. ¹HNMR (500 MHz, CDCl₃) δ: 11.75 (br s, 1H), 4.02 (m, 1H), 3.00 (m, 1H), 2.90-2.66 (m, 6H).

### Intermediate 8

2-[(Tert-butoxycarbonypamino]-5,6-dihydro-4H-cyclopenta [α] [1,3] thiazole-4-carboxylic acid (i-8)

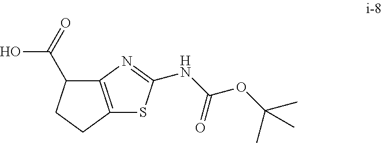

Step A: Ethyl 2-amino-5,6-dihydro-4H-cyclopenta [α] [1,3] thiazole-4-carboxylate

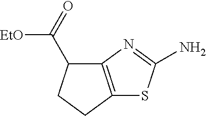

Prepared in the same manner as intermediate (i-7) replacing the thioacetamide in Step A with thiourea. ¹HNMR (500 MHz, CDCl₃) δ: 5.30 (br s, 2H), 4.21 (q, J=7.0, 2H), 3.81 (m, 1H), 2.91 (m, 1H), 2.78 (m, 1H), 2.66 (m, 2H), 1.30 (t, J=7.0, 3H).

Step B: Ethyl 2-[(tert-butoxycarbonyl)amino]-5,6-dihydro-4H-cyclopenta [α] [1,3] thiazole-4-carboxylate

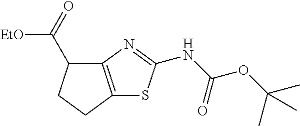

To a solution of 230 mg (1.08 mmol) of ethyl 2-amino-5,6-dihydro-4H-cyclopenta [α] [1,3] thiazole-4-carboxylate in dichloromethane (5 mL) (from step A) was added di-cert butyldicarbonate (236 mg, 1.08 mmol), triethylamine (0.15 mL, 1.08 mmol) and DMAP (13 mg, 0.11 mmol) and the resulting mixture stirred at room temperature for 2 h. Mixture washed with 1N HCl (10 mL), sat. NaCl (5 mL), dried over MgSO₄, filtered and evaporated. Residue purfied by MPLC (Biotage Horizon: FLASH 25+S) eluent: 100% Hexanes (100 mL), gradient 0-15% EtOAc in Hexanes (900 mL) then 15% EtOAc in Hexanes (500 mL) to give the title compound (160 mg, 47%) as a white foam, ¹HNMR (500 MHz, CDCl₃) δ: 9.23 (br s, 1H), 4.17 (q, J=7.1 Hz, 2H), 3.95 (t, J=6.6 Hz, 1H), 3.04 (m, 1H), 2.86 (m, 1H), 2.76 (m, 2H), 1.55 (s, 9H), 1.23 (t, J=7.1 Hz, 3H).

Step C: 2-[(Tert-butoxycarbonyl)amino]-5,6-dihydro-4H-cyclopenta [α] [1,3] thiazole-4-carboxylic acid (i-8)

Prepared from ethyl 2-[(Cert-butoxycarbonyl)amino]-5,6-dihydro-4H-cyclopenta [α] [1,3] thiazole-4-carboxylate (from step B) using a procedure analogous to that found in intermediate (i-7) step B. ¹HNMR (500 MHz, CDCl₃) δ: 3.96 (m, 1H), 3.06 (m, 1H), 2.88 (m, 2H), 2.71 (m, 1H), 1.55 (s, 9H).

### Intermediate 9

2-(4-Fluorophenyl)-5,6-dihydro-4H-cyclopenta [α] [1,3] thiazole-4-carboxylic acid 0-9)

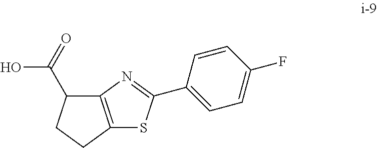

Prepared using procedures analogous to those found in intermediate 7 (i-7) replacing thioacetamide with 4-fluorothiobenzamide in step A. ¹HNMR (500 MHz, DMSO-d6) δ: 7.90 (m, 2H), 7.29 (t, J=8.7, 2H), 3.81 (m, 1H), 2.99 (m, 1H), 2.86 (m, 1H), 2.70-2.58 (m, 2H).

### Intermediate 10

2-Methyl-4,5,6,7-tetrahydro-1,3-benzothiazole-4-carboxylic acid (i-10)

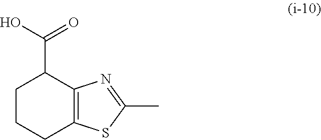

Step A: Ethyl 2-methyl-4,5,6,7-tetrahydro-1,3-benzothiazole-4-carboxylate

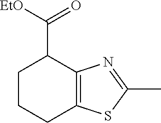

To a solution of ethyl 2-oxocyclohexanecarboxylate (15 g, 88 mmol) in anhydrous diethyl ether (40 mL) cooled at 0° C. was added bromine (4.5 mL, 88 mmol) dropwise over 15 mins. After complete addition mixture allowed to warm to room temp over 90 min. Mixture diluted with EtOAc (100 mL) and washed with sat. NaHCO₃, sat. NaCl, dried over MgSO₄, filtered and evaporated. Residue taken up in ethanol (100 mL) and thioacetamide (6.6 g, 88 mmol) added. Mixture stirred at room temp for 1 h then at reflux overnight. Mixture evaporated and residue partitioned between sat. NaHCO₃ and DCM. Organic layer dried over MgSO₄, filtered and evaporated. Residue purified by MPLC (Biotage Horizon: FLASH 65i) eluent: 100% Hexanes (500 mL), gradient 0 to 25% EtflAc in Hexanes (1200 mL) then 25% EtOAc in Hexanes (1200 mL) to give the title compound (6.14 g, 31%) as a pale orange oil. ¹HNMR (500 MHz, CDCl₃) δ: 4.22 (q, J=7.1, 2H), 3.84 (t, J=5.5, 1H), 2.80 (m, 1H), 2.73 (m, 1H), 2.65 (s, 3H), 2.18 (m, 1H), 2.11-1.95 (m, 2H), 1.85 (m, 1H), 1.29 (t, J=7.1, 3H).

Step B: 2-Methyl-4,5,6,7-tetrahydro-1,3-benzothiazole-4-carboxylic acid (i-10)

Prepared from ethyl 2-methyl-4,5,6,7-tetrahydro-1,3-benzothiazole-4-carboxylate (from step A) according to the procedure outlined in intermediate (i-7) step B. ¹HNMR (500 MHz, CDCl₃) δ: 9.26 (br s, 1H), 3.81 (q, J=7.3 and 5.9, 1H), 2.75 (m, 2H), 2.68 (s, 3H), 2.24 (m, 1H), 2.18-2.01 (m, 2H), 1.82 (m, 1H).

### Intermediate 11

2-[(Tert-butoxyearbonyl)amino]-4,5,6,7-tetrahydro-1,3-benzothiazole-4-carboxylic acid (i-11)

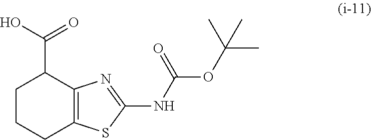

Step A: Ethyl 2-amino-4,5,6,7-tetrahydro-1,3-benzothiazole-4-carboxylate

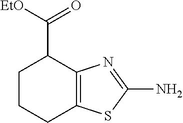

Prepared according to the procedures outlined in intermediate 10 (i-10) step A replacing thioacetamide with thiourea. ¹HNMR (500 MHz, DMSO-d6) δ: 9.28 (br s, 2H), 4.11 (q, J=7.3, 2H), 3.71 (t, J=5.0, 1H), 2.57-2.39 (m, 2H), 1.90 (m, 2H), 1.78 (m, 1H), 1.59 (m, 1H), 1.17 (t, J=7.3, 3H).

Step B: 2-[(Tert-butoxycarbonyl)amino]-4,5,6,7-tetrahydro-13-benzothiazole-4-carboxylic acid (i-11)

Prepared from ethyl 2-amino-4,5,6,7-tetrahydro-1,3-benzothiazole-4-carboxylate (from step A) according to the procedures outlined in intermediate 8 (i-8) steps B and C. ¹HNMR (500 MHz, CDCl₃) δ: 3.70 (t, J=5.2, 1H), 2.74 (m, 1H), 2.64 (m, 1H), 2.25 (m, 1H), 2.10-1.94 (m, 2H), 1.87 (m, 1H), 1.55 (s, 9H).

### Intermediate 12

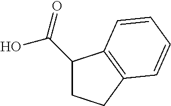

Indan-1-carboxylic acid (i-12)

Prepared according to the literature procedure Journal of Organic Chemistry (2000), 65(4), 1132-1138.

### Intermediate 13A and Intermediate 13B

Tert-butyl (2S, 5R)-2-(4-aminobenzyl)-5-[(R)-hydroxy(phenyl)methyl]pyrrolidine-1-carboxylate (i-13 a);
Tert-butyl (2R, 5R)-2-(4-aminobenzyl)-5-[(R)-hydroxy(phenyl)methyl]pyrrolidine-1-carboxylate (i-13 b)

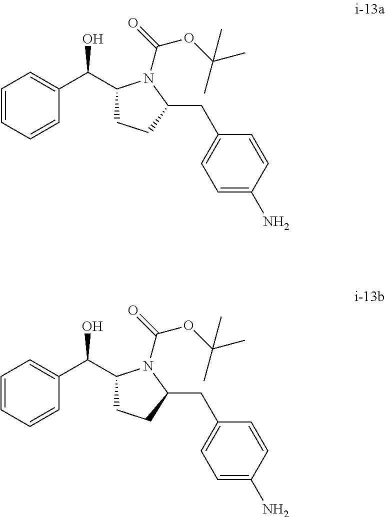

Step A: Tert-butyl (4R, 5R)-2,2-dimethyl-4-[(1E)-3-oxoprop-1-en-1-yl]-5-phenyl-1,3-oxazolidine-3-carboxylate

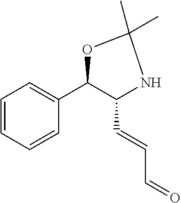

To a solution of tert-butyl (4S, 5R)-4-formyl-2,2-dimethyl-5-phenyl-1,3-oxazolidine-3-carboxylate (20.9, 89.1 mmol) in CH₂Cl₂ (150 mL) was added (triphenylphosphoranylidene) acetaldehyde (27.1 g, 89.1 mmol) and the resulting mixture was stirred at ambient temperature for 40 h. After removal of ⅓ of the solvent, hexanes was generously added and the resulting solid was filtered off. Flash chromatography on a Biotage Horizon® system (silica gel, 0 to 20% ethyl acetate in hexanes gradient then 20% ethyl acetate in hexanes) gave 16.3 g (72%) of the title compound as a yellow oil. LC/MS 354.3 (M+23).

Step B: Tert-butyl (4R, 5R)-2,2-dimethyl-4-(3-oxopropyl)-5-phenyl-1,3-oxazolidine-3-carboxylate

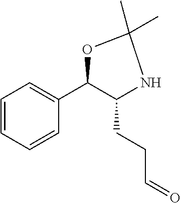

To a solution of tert-butyl (4R, 5R)-2,2-dimethyl-4-[(1E)-3-oxoprop-1-en-1-yl]-5-phenyl-1,3-oxazolidine-3-carboxylate (19.6 g, 59.1 mmol) (from Step A) in acetone (150 mL) was added 1.9 g of 10% Pd/C and the resulting suspension was stirred under a hydrogen balloon at ambient temperature for 24 h. The solid was filtered off on celite and the filtrate concentrated under vacuum. The residue was purified by flash chromatography on a Biotage Horizon® system (silica gel, 0 to 20% ethyl acetate in hexanes gradient then 20% ethyl acetate in hexanes) to afford 11.5 g (58%) of the title compound as a colorless oil. LC/MS 356.3 (M+23).

Step C: Tert-butyl (4R, 5R)-2,2-dimethyl-4-[(3E)-4-(4-nitrophenyl)but-3-en-1-yl]-5-phenyl-1,3-oxazolidine-3-carboxylate and tert-butyl (4R, 5R)-2, 2-dimethyl-4-[(3Z)-4-(4-nitrophenvI)but-3-en-1-yl]-5-phenyl-1,3-oxazolidine-3-carboxylate

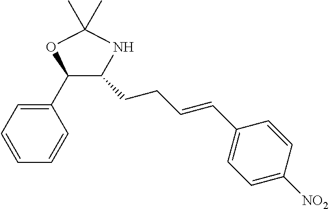

To a solution of tert-butyl (4R, 5R)-2, 2-dimethyl-4-(3-oxopropyl)-5-phenyl-1,3-oxazolidine-3-carboxylate (10.0 g, 30.0 mmol) from Step B in CH₂Cl₂ (200 mL) was added (4-nitrobenzyl)triphenyl-phosphonium bromide (21.5 g, 45,0 mmol) followed by Et₃N (8.36 mL, 60.0 mmol). The red reaction mixture was stirred at ambient temperature for 48 h. Hexane (200 mL) was poured into the reaction mixture and the solid was filtered off. Flash chromatography on a Biotage Horizon® system (silica gel, 0 to 10% ethyl acetate in hexanes gradient then 10% ethyl acetate in hexanes) afforded 10.7 g (79%) of the title compounds (cis trans mixture) as pale yellow foam. LC/MS 475.4 (M+23).

Step D: Tert-butyl (2R, 5S)-2-[(R)-hydroxy(phenyl)methyl]-5-(4-nitrobenzyl)pyrrolidine-1-carboxylate and tert-butyl (2R, 5R)-2-[(R)-hydroxy(phenyl)methyl]-5-(4-nitrobenzyl)pyrrolidine-1-carboxylate

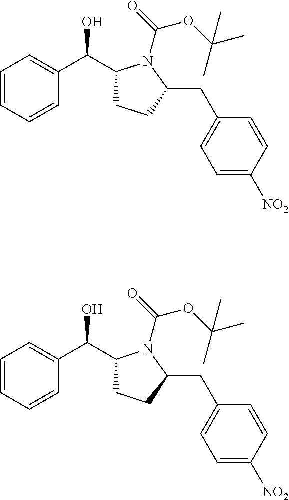

To a solution of the above cis/trans mixture (7.86 g, 17.4 mmol) from Step C in ethyl acetate (100 mL) was added 50 mL of 2N HCl solution and the resulting mixture was stirred at ambient temperature for 2 h then heated to 45° C. for 3 h. The volatiles were removed under reduced pressure. The resulting white solid was dissolved in N, N-dimethylformamide (100 mL) and 15.1 mL (86.7 mmol) of ¹Pr₂Net was added. The reaction mixture was stirred at ambient temperature for 7 h. Di-tert-butyl dicarbonate (4.55 g, 20.8 mmol) was then added and the reaction mixture was stirred at ambient temperature overnight. Water (200 mL) was added and it was extracted with ethyl acetate (200 mL×3). The combined organic layers were dried over Na₂SO₄, filtered and concentrated under reduced pressure. The residue was purified by flash chromatography on a Biotage Horizon® system (silica gel, 0 to 30% ethyl acetate in hexanes gradient) to afford 1.61 g (22%) of the title compounds tert-butyl-(2R, 5S)-2-[(R)-hydroxy(phenyl)methyl]-5-(4-nitrobenzyl)pyrrolidine-1-carboxylate-(cis) and 3.9 g (54%) of tert-butyl (2R, 5R)-2-[(R)-hydroxy(phenyl)methyl]-5-(4-nitrobenzyl)pyrrolidine-1-carboxylate (trans). LC/MS 435.4 (M+23).

Step E: Tert-butyl (2S, 5R)-2-(4-aminobenzyl)-5-[(R)-hydroxy(phenyl)methyl]pyrrolidine-1-carboxylate (i-13a)

To a solution of the above (cis) tert-butyl (2R, 5.5)-2-[(R)-hydroxy(phenyl)methyl]-5-(4-nitrobenzyl)pyrrolidine-1-carboxylate (1.51 g, 3.66 mmol) from Step D in ethanol (20 mL) was added 0.15 g of 10% Pd/C and the resulting suspension was stirred under a hydrogen balloon at ambient temperature for 5 h. Filtration through celite and removal of the solvent gave 1.40 g (100%) of the title compound as white foam which was used without further purification. LC/MS 405.3 (M+23).

Step F: Tert-butyl (2R, 5R)-2-(4-aminobenzyl)-5-[(R)-hydroxy(phenyl)methyl]pyrrolidine-1-carboxylate (i-13b)

To a solution of (trans) tert-butyl (2R, 5R)-2-[(R)-hydroxy(phenyl)methyl]-5-(4-nitrobenzyl)pyrrolidine-1-carboxylate (3.90 g, 9.46 mmol) from Step D in ethanol (40 mL) was added 0.4 g of 10% Pd/C and the resulting suspension was stirred under a hydrogen balloon at ambient temperature for 6 h. The solid was filtered off through celite. After removal of the solvent, flash chromatography on a Biotage Horizon® system (silica gel, 0 to 30% ethyl acetate in hexanes gradient then 30% ethyl acetate in hexanes) afforded 2.30 g (64%) of the title compound as a white foam. LC/MS 405.3 (M+23).

### Intermediate 14

(2S)-1-(1,3-benzothiazol-2-yl)pyrrolidine-2-carboxylic acid (i-14):

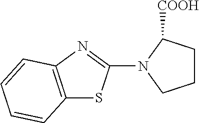

To a solution of 28 mg (0.24 mmol) of L-Proline in N, N-dimethylformamide (3 mL) at ambient temperature was added 51 mg (0.24 mmol) of 2-bromobenzothiazole, 100 mg (0.72 mmol) of potassium carbonate, and 6 mg (0.03 mmol) of copper iodide. The reaction mixture was stirred at 100° C. overnight. It was then filtered and purified by reverse-phase HPLC (TMC Pro-Pac C18; 0-60% 0.1% trifluoroacetic acid in acetonitrile/0.1% trifluoroacetic acid in water gradient). The pure fractions were lyophilized overnight to give 35 mg 60% of the title compound as a light brown solid. ¹H NMR (DMSO-d₆): δ 7.78 (d, J=8.0 Hz, 1H), 7.45 (d, J=8.0 Hz, 1H), 7.28 (t, J=7.8 Hz, 1H), 7.08 (t, J=7.8 Hz, 1H), 4.48 (d, J=7.3 Hz, 1H), 3.52-3.61 (m, 2H), 2.37 (m, 1H), 2.01-2.11 (m, 3H). LC/MS 249.3 (M+1)

### Intermediate 44

Preparation of [(6,S)-4-oxo-4,6,7,8-tetrahydropyrrolo[1,2-]pyrimidine-6-carboxylic acid (i-44)

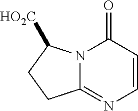

Step A: Methyl [6(S)-4-oxo-4,6,7,8-tetrahydropyrrolo[1,2-]pyrimidine-6-carboxylate

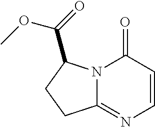

Methyl (2S)-5-methoxy-3,4-dihydro-2H-pyrrole-2-carboxylate (4.19 g, 26.6 mmol) and 3-azatricyclo[4.2.1.0.^{2,5}]non-7-en-4-one (2.4 g, 17.8 mmol) was heated at 110° C. overnight. Purification using a Biotage Horizon® system (0-100% ethyl acetate/hexanes mixture) gave the title compound methyl [6(S)-4-oxo-4,6,7,8-tetrahydropyrrolo[1,2-^{α}]pyrimidine-6-carboxylate and intermediate methyl (75)-9-oxo-3,8-diazatetracyclo[9.2.1.0^{2,10}.0^{4,8}]tetradeca-3,12-diene-7-carboxylate. The intermediate was heated at 150° C. for 45 min to afford the title compound without further purification. LC/MS 195.2 (M+1).

Step B: [(6S)-4-oxo-4,6,7,8-tetrahydropyrrolo[1,2-]pyrimidine-6-carboxylic acid

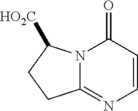

Methyl [6(S)-4-oxo-4,6,7,8-tetrahydropyrrolo[1,2-^{α}]pyrimidine-6-carboxylate (9.95 g, 51.2 mmol) in tetrahydrofuran (60 mL), methanol (40 mL) and a solution of lithium hydroxide (3.32g, 77 mmol) in water (40 mL) was stirred at ambient temperature for 1 h. 2 N hydrochloric acid (38.5 mL) was added to neutralize the reaction mixture which was then directly purified by reverse phase HPLC (TMC Pro-Pac C18; 0-40% 0.1% trifluoroacetic acid in acetonitrile/0.1% trifluoroacetic acid in water gradient). The O-alkylation product was eluted fast. The pure fractions were collected and lyophilized overnight to afford the title compound as a pale yellow solid. ¹HNMR (DMSO-d₆): δ 7.89 (d, J=6.6 Hz, 1H), 6.24 (d, J=6.6 Hz, 1H), 4.92 (dd, J=10.0, 3.1 Hz, 1H), 3.12-2.99 (m, 2H), 2.52 (m, 1H), 2.11 (m, 1H). LC/MS 181.2 (M+1).

### Intermediate 46

(3S)-5-Oxo-1,2,3,5-tetrahydroindolizine-3-carboxylic acid (i-46)

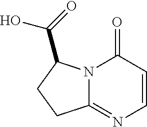

Step A: (3S,9S)-5-Oxo-1,2,3,5,6,8a-hexahydroindolizine-3-carboxylic acid methyl ester

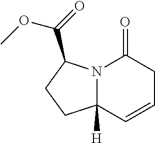

This intermediate was prepared according to the procedures found in: Hanessian, S.; Sailes, H.; Munro, A.; Therrien, E. J. Org. Chem. 2003, 68, 7219 and Vaswani, R. G.; Chamberlin, R. J. Org. Chem. 2008, 73, 1661.

Step B: Methyl (3S)-5-oxo-1,2,3,5-tetrahydroindolizine-3-carboxylate

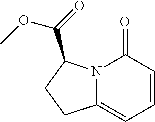

To a stirred solution of 0.850 g (4.06 mmol) of (3S,9S)-5-oxo-1,2,3,5,6,8a-hexahydroindolizine-3-carboxylic acid methyl ester from step A above in 50 mL of dichloromethane was added 6.30 g (72.5 mmol) of manganese(IV) oxide and the resulting mixture was stirred for 12 h at reflux. The reaction was cooled to ambient temperature, filtered through a pad of Celite®, and the solid was then washed with 100 mL of dichloromethane. The filtrate was evaporated to dryness in vacua and the residue was purified by silica gel chromatography eluting with 10-50% ethyl acetate in hexanes gradient to afford the title compound as a clear gum (0.47 g, 55% yield). LC-MS: m/z (ES) 194 (MH)⁺.

Step C: (3S)-5-Oxo-1,2,3,5-tetrahydroindolizine-3-carboxylic acid

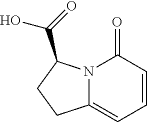

To a stirred solution of 0.200 mg (1.00 mmol) of methyl (35)-5-oxo-1,2,3,5-tetrahydroindolizine-3-carboxylate from step B above in 3 mL of THF was added 1.5 mL (1.5 mmol) of a 1.0 M aqueous LiOH solution. The resulting mixture was stirred for 2 h at ambient temperature then quenched with 2.0 mL (2.0 mmol) of a 1.0 M aqueous solution of hydrogen chloride. All volatiles were evaporated in vacuo and the aqueous phase was extracted with a 30% IPA in chloroform mixture (3×5 mL). The combined extract were washed with brine, dried over magnesium sulfate, filtered, and evaporated in vacuo to afford the title compound as a white solid (0.17 g, 92%). ¹H NMR (500 MHz, CD₃OD): δ 7.53 (dd, J=8.9, 7.1 Hz, 1H), 6.38-6.35 (m, 2H), 5.11 (dd, J=9.7, 2.7 Hz, 1H), 3.23-3.12 (m, 2H), 2.62-2.53 (m, 1H), 2.35-2.30 (m, 1H). LC-MS: ink (ES) 180 (MH)⁺.

### Intermediate 56

2-(3-methyl-I H-1,2,4-triazol-1-yl)propanoic acid (i-56)

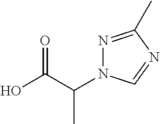

Step A: Tert-butyl 2-(3-methyl-1H-1.2,4-triazol-1-yl)propanoate

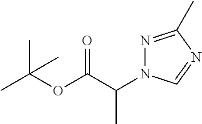

To a solution of 3-methyl-1H-1,2,4-triazole (7.3 g, 88 mmol) in DMF (75 mL) was added K₂CO₃ (60.7 g, 439 mmol) and 2-bromopropionic acid tert-butyl ester (14.6 mL, 88 mmol). The reaction was stirred at room temperature overnight. The mixture was diluted with EtOAc (500 mL), washed with water (x 3) then brine. Dried over MgSO4 and concentrated. The residue was purified by column chromatography on silica gel, eluting with EtOAc/isohexane (20 to 100%) to give 13 g of crude product as a 3:1 mixture of regioisomers. The mixture was purified by Chiralcel OD with a gradient from 4% to 30% IPA/Heptane. Then the first two peaks were separated with Chiracel OD column isocratically eluting with 4% IPA/Heptane. The second peak was collected as the desired single stereoisomer (R or S) (243-methyl-1H-1,2,4-triazol-1-yl)propanoic acid tert-butyl ester) (3.5 g, 19%). ¹H-NMR (500 MHz, CDCl₃) δ 8.05 (s, 1H), 4.90 (q, J=7 Hz, 1H), 2.35 (s, 3 H), 1.72 (d, J=7 Hz, 3 H), 1.40 (s, 9 H). ESI-MS calculated for C₁₀H₁₇N₃O₂: Exact Mass: 211.13; Found 156.05 (-tBu).

Step B: 2(3-methyl-1H-1,2,4-triazol-1-yl)propanoic acid

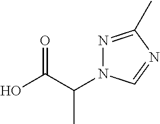

Tert-butyl 2-(3-methyl-1H-1,2,4-triazol-1-yl)propanoate (1.0 g, 4 7 mmol) was dissolved in 4 M HCl in dioxane (100 mL) and stirred at room temperature overnight. The product was concentrated under reduced pressure and dried under high vacuum to give (R or S) tert-butyl 2-(3-methyl-1H-1,2,4-triazol-1-yl)propanoate as the HCl salt (850 mg). ESI-MS calculated for C₆H₉N₃O₂: Exact Mass: 155.07; Found 156.05.

### Compound 1

2-(2-Amino-1,3-thiazol-4-yl)-N-[4-({(5R)-[(R)-hydroxy(phenyl)methyl]pyrrolidin yl}methyl)phenyl]acetamide

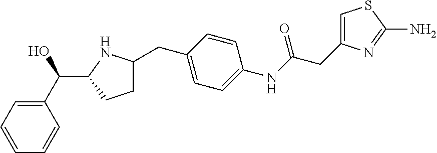

Step A: Tert-butyl (5R)-2-(4-{[(2-amino-1,3-thiazol-4-yl)acetyl]amion}benzyl)-5-[(R)-{[tert-butyl(dimethyl)silyl]oxy}(phenyl)methyl]pyrrolidine-1-carboxylate

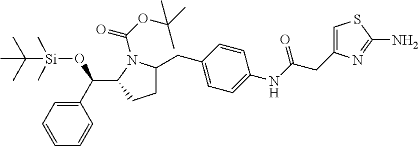

To a solution of 10 mg (5:1 mixture cis/trans, 0.02 mmol) of tert-butyl(5R)-2-(4-aminobenzyl)-5-[(R)-{[tert-butyl(dimethyl)silyl]oxy}(phenyl)methyl]pyrrolidine-1-carboxylate (i-3) and (2-amino-1,3-thiazol-4-yl)acetic acid (3.18 mg, 0.02 mmol) in 0.5 mL anhydrous DMF was added a 0.5 M solution of HOAt in DMF (0.04 mL, 0.02 mmol) followed by EDC (5.8 mg, 0.03 mmol) and DMA (3.5 μL, 0.02 mmol). The resulting mixture was stirred at room temperature under nitrogen atmosphere for 16 h. The mixture was washed with water and extracted with dichloromethane (2×2 mL). The organics were combined, dried over sodium sulfate, filtered and concentrated in vacuo. The residue was purified by preparative TLC plate (500 uM) eluting with 5% MeOH in dichloromethane to afford the product (10.3 mg, 81%). m/z (ES) 637 (MH)⁺, 659 (MNa)⁺.

Step B: 2-(2-Amino-1,3-thiazol-4-yl)-N-[4-({(5R)-[(R)hydroxy(phenyl)methyl]pyrrolidinyl}methyl)phenyl]acetamide

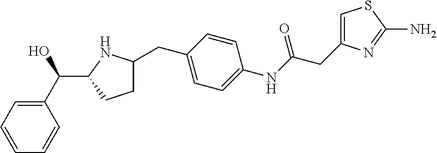

To a solution of 7 mg (0.01 mmol) of tert-butyl (5R)-2-(4-{[(2-amino-1,3-thiazol-4-yl)acetyl]amion}benzyl)-5-[(R)-{[tert-butyl(dimethyl)silyl]oxy}(phenyl)methyl]pyrrolidine-1-carboxylate in 0.20 mL methanol (from Step A) was added 0.20 mL cone. HCl and the reaction mixture stirred at room temperature for 1 h. Azeotrop with toluene (2×) to remove water. The residue was taken up in acetonitrile/water/MeOH (9:1:1) and purified on the Gilson HPLC eluting with a 0-50% gradient of acetonitrile/water with 0.05% TFA buffer. The fractions containing the product were combined, frozen, and lyophilized to give a white foam (3.3 mg, 71%). m/z (ES) 423 (MH)⁺. (˜5:1 mixture) ¹HNMR (500 MHz, CD₃OD) δ: 7.56 (br d, J=8.2 Hz, 2H), 7.44 (d, 7.8 Hz, 2H), 7.39 (t, J=7.6 Hz, 2H) 7.35-7.32 (m, 0.8H) 7.32-7.29 (m, 0.2H minor isomer), 7.26 (d, J=8.0 Hz, 1.7H), 7.14 (d, J=8.1 Hz, 0.3H minor isomer) 6.67 and 6.66 (br s, 0.2/0.8H, totaling 1H). 4.72 (d, J=8.5 Hz, 1H), 3.80-3.70 (m, 4H) 3.14 (dd, J=6.1, 13.8 Hz, 1H), 2.95 (dd, J=9.1, 13.8 Hz, 1H), 2.08-2.00 (m, 1H), 1.86-1.74 (m, 3H).

Using the Biological Assays described herein, the human β3 functional activity of Compound 1 was determined to be between 1 to 10 nM.

### Compound 2

2-(2-Amino-1,3-thiazol-4-yl)-N-[4-({(2S, 5R)-[(R)-hydroxy(phenyl)methyl]pyrrolidin yl}methyl)phenyl]acetamide

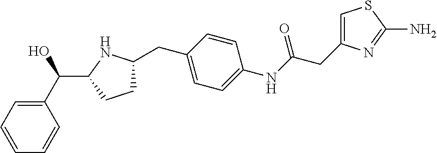

Step A: Tert-butyl (2S, 5R)-2-(4-{[(2-amino-1,3-thiazol-4-yl)acetyl]amion}benzyl)-5-[(R)-{[tert-butyl(dimethyl)silyl]oxy}(phenyl)methyl]pyrrolidine-1-carboxylate

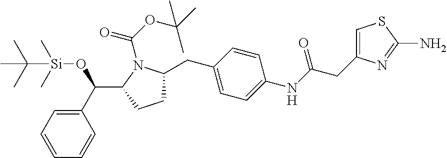

The title compound was prepared from tert-butyl (2S, 5R)-2-(4-aminobenzyl)-5-[(R)-{[tert-butyl(dimethyl)silyl]oxy}(phenyl)methyl]pyrrolidine-1-carboxylate (i-4a) and (2-amino-1,3-thiazol-4-yl)acetic acid according to the procedure of Compound 1, step A. The crude product was purified by preparative TLC plate eluting with 5% MeOH in dichloromethane to afford the product (4.1 mg, 21%). m/z (ES) 637 (MH)⁺, 659 (MNa)⁺.

Step B: 2-(2-Amino-1,3-thiazol-4-yl)-N-[4-({(2S, 5R)-[(R)hydroxy(phenyl)methyl]pyrrolidinyl}methyl)phenyl]acetamide

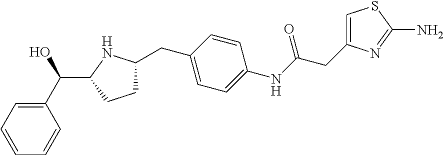

The title compound was prepared from 4 mg of text-butyl (2S, 5R)-2-(4-{[(2-amino-1,3-thiazol-4-yl)acetyl]amion}benzyl)-5-[(R)-{[tert-butyl(dimethyl)sily]oxy}(phenyl)methyl]pyrrolidine-1-carboxylate (from Step A) according to the procedure of Compound 1, step B. The crude product was purified on the Gilson HPLC eluting with a 0-50% gradient of acetonitrile/water with 0.05% TPA buffer. The fractions containing the product were combined, frozen, and lyophilized to give a white foam (3.3 mg, 71%). m/z (ES) 423 (MH)⁺. ¹HNMR (500 MHz, CD₃OD) δ: 7.55 (br d, J=8.2 Hz, 2H), 7.44 (d, 7.8 Hz, 2H), 7.39 (t, J=7.6 Hz, 2H) 7.35-7.33 (m, 1H), 7.25 (d, J=8.0 Hz, 2H), 6.65 (br s,1H). 4.72 (d, J=8.5 Hz, 1H), 3.80-3.72 (m, 4H) 3.14 (dd, J=6.1, 13.8 Hz, 1H), 2.96 (dd, J=9.1, 13.8 Hz, 1H), 2.07-2.00 (m, 1H), 1.85-1.73 (m, 3H).

Using the Biological Assays described herein, the human β3 functional activity of Compound 2 was determined to be between 1 to 10 nM.

### Compound 3

2-Amino-N-[4-{((2S, 5R -5-[(R)-hydroxy(phenyl)methyl]pyroolidin-2-yl)methyl)phenyl]-5,6-dihydro-4H-cyclonenta [α] [1,3] thiazole-4-carboxamide

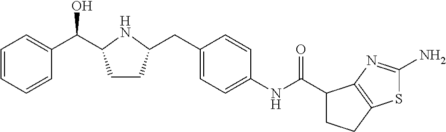

Step A: Tert-butyl-(2S, 5R)-2-(4[({2-[(tert-butoxycarbonyl)amino]-5,6-dihydro-4H-cyclopenta [α] [1,3] thiazol-4-yl}carbonyl)amino]benzyl}-5-[(R)-hydroxy(phenyl)methyl]pyrrolidine-1-carboxylate

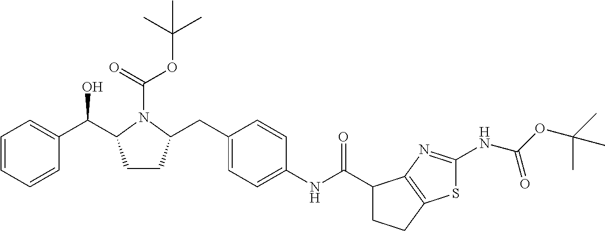

To a solution of 220 mg (0.58 mmol) of tert-butyl (2S, 5R)-2-(4-aminobenzyl)-5-[(R)-hydroxy(phenypmethyl] pyrrolidine-1-carboxylate (i-13a) and 164 mg (0.58 mmol) of 2-[(tert-butoxycarbonypamino]-5,6-dihydro-4H-cyclopenta [α] [1,3] thiazole-4-carboxylic acid (i-8) in anhydrous DMF (5 mL) was added EDC (165 mg, 0.86 mmL), HOBt (132 mg, 0.86 mmol) and Hunig's Base (0.3 mL, 1.7 mmol) and the resulting mixture stirred at room temperature overnight. Poured into water (50 mL) and extracted with EtOAc (3×30 mL), combined EtOAc layers washed with water (2×50 mL), sat. NaCl (25 mL), dried over MgSO₄, filtered and evaporated. Residue purified by MPLC (Biotage Horizon: FLASH 25+M) eluent: 100% Hexanes (100 mL), gradient 0 to 35% EtOAc in Hexanes (750 mL), then 35% EtOAc in Hexanes (600 mL). Diastereoisomers separated by chiral HPLC on AD column (eluent:25% IPA in Heptane) first eluting isomer (134 mg, 36%) second eluting isomer (126 mg, 34%) both as white foams.

Step B: 2-Amino-N-[4-{((2S, 5R)-5-[(R)hydroxy(phenyl)methyl]pyroolidin-2-yl)methyl)phenyl]-5,6-dihydro-4H-cyclopenta [α] [1,3] thiazole-4-carboxamide

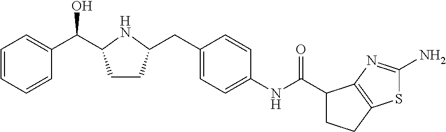

To a solution of 126 mg (0.19 mmol) of tert-butyl-(2S, 5R)-2-(4[({2-[(tert-butoxycarbonyl)amino]-5,6-dihydro-4H-cyclopenta [α] (1,3] thiazol-4-yl}carbonyl)aminolbenzy}-5-[(R)-hydroxy(phenyl)methyl]pyrrolidine-1-carboxylate (from step A, second eluting isomer) in DCM (3 mL) was added trifluoroacetic acid (3.0 mL, 38 mmol) the resulting mixture stirred at room temperature for 4 h. The mixture was evaporated and passed through an SCX cartridge eluting with 2 M NH₃ in methanol to free up the base. Product purified by PREP-TLC 2×[20×20 cm×1000 micron] eluent: 15% MeOH in DCM+1% NH₄OH and product lyophilized to give the title compound (65 mg, 75%) as a white fluffy solid. m/z (ES) 449 (MH)⁺. ¹HNMR (500 MHz, DMSO-d6) δ: 10.00 (s, 1H), 7.51 (d, J=8.2, 2H), 7.30 (m, 4H), 7.21 (t, J=6.9, 1H), 7.12 (d, J=8.2, 2H), 6.86 (s, 1H), 4.23 (d, J=7.3, 1H), 3.78 (m, 1H), 3.21 (m, 1H), 3.10 (m, 1H) 2.78 (m, 1H), 2.66 (m, 2H), 2.57 (m, 2H), 2.49 (m, 1H), 1.59 (m, 1H), 1.40 (m, 1H), 1.39 (m, 2H).

Product from Step A [first eluting isomer] (134 mg, 0.207 mmol) was deprotected in similar fashion to give the title compound (44 mg, 48%) as a white fluffy solid. m/z (ES) 449 (MH)⁺.

Using the Biological Assays described herein, the human β3 functional activity of Compound 3 was determined to be less than 1 nM.

### Compounds 4-10

Using procedures similar to those described above, Compounds 4-10 were prepared from the appropriate starting materials.

Using the Biological Assays described herein, the human β3 functional activity of each compound was determined and shown in the following table.

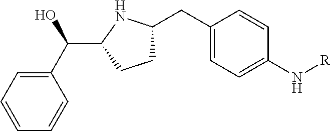

| | | | | HU- |
|---|---|---|---|---|
| Com- | | | | MAN |
| pound | | | MS | β3 |
| Num- | | | (ES) | BIND- |
| ber | R | MW | (MH)⁺ | ING |
| 4 | 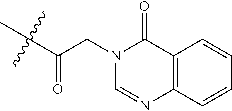 | 468.56 | 469.50 | 1-10 NM |
| 5 | 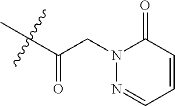 | 418.49 | 419.24 | 1-10 NM |
| 6 | 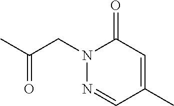 | 432.53 | 433.50 | 1-10 NM |
| 7 | 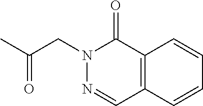 | 468.56 | 469.52 | 1-10 NM |
| 8 | 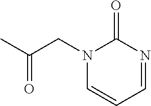 | 418.50 | 419.48 | 1-10 NM |
| 9 | 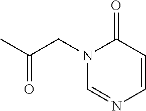 | 418.50 | 419.48 | 1-10 NM |
| 10 | 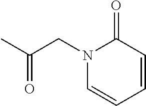 | 417.51 | 418.50 | 1-10 NM |

### Compound 11

N-(4-((2S,5R)-5-((R)-hydroxy(phenyl)mehtyl)pyrrolidin-2-yl)methyl)phneyl)-2-(3-methyl-1H-1,2,4-triazol-1-yl)propanamide

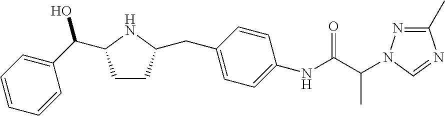

A mixture of i-13a (2.00 g, 5.23 mmol), 243-methyl-1H-1,2,4-triazol-1-yl)propanoic acid i-56 (1.00 g, 5.23 mmol), HOAt (1.307 mL, 0.784 mmol), and EDC (2.005 g, 10.46 mmol) in DMF (20 mL) was stirred at room temperature for 10 min, The reaction mixture was quenched with aqueous sodium bicarbonate and extracted with EtOAc. The crude product was purified by column chromatography (0-3% MeOH (10% NH4OH) in DCM. After evaporation, the product was further purified by chiral HPLC (AD column, 30% TPA/Heptanes) to give the pure boc protected intermediate, which was dissolved in a minimal volume of dioxane and 4 M HCl in dioxane was added. After 2 h at room temperature, the reaction mixture was concentrated under reduced pressure to give the HCl salt of the title compound. Basic reverse phase HPLC (0.1% NH₄OH in H₂O, MeCN) yielded the desired free base of the title compound. ¹H-NMR (500 MHz, CD₃OD) δ 8.51 (s, 1H), 7.49 (d, J=13 Hz, 2 H) 7.35-7.29 (m, 4 H), 7.26-7.20 (m ,4 H), 5.20 (q, J=7.5 Hz, 1H), 4.20 (d, J=7.5 Hz, 1H), 3.27-3.22 (m, 2 H), 2.80-2.72 (m, 2 H), 2.34 (s, 3 H), 1.82 (d, J=7.5 Hz, 3 H), 1.79-173 (m, 1H), 1.52-1.48 (m, 3 H). EST-MS calculated for C₂₄H₂₉N₅O₂: Exact Exact Mass: 419.23, found 420.35.

Using the Biological Assays described herein, the human β3 functional activity of Compound 11 was determined to be between 1 to 10 nM.

### Compounds 12 and 13

(3S)-N-[4-({(2S,5R)-5-[(R)-hydroxy(phenyl)methyl]pyrrolidin-2-yl}methyl)phenyl]-5-oxo-1,2,3,5-tetrahydroindolizine-3-carboxamide (Compound 12) and (3R)-N-[4-({(25,5R)-5-[(R)-hydroxy(phenyl)methyl]pyrroliclin-2-yl}methyl)phenyl]-5-oxo-1,2,3,5-tetrahydroindolizine-3-carboxamide (Compound 13)

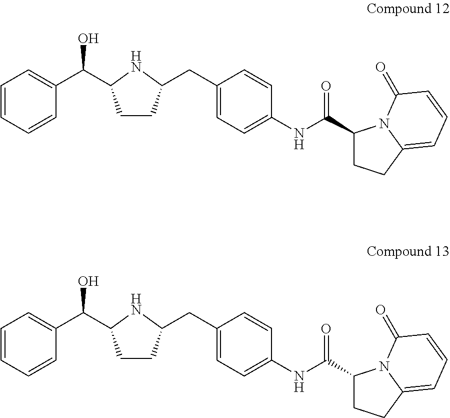

Step A: Tert-butyl (2R,5S)-2-[(R)-hydroxy(phenyl)methyl]-5-[4-({[(3S)-5-oxo-1,2,3,5-tetrahydroindolizin-3-yl]carbonyl}amino)benzyl]pyrrolidine-1-carboxylate (isomer 1) and tert-butyl (2R,5S)-2-[(R)-hydroxy(phenyl)methyl]-5-[4-({[(3R)-5-oxo-1,2,3,5-tetrahydroindolizin-3-yl]carbonyl}amnino)benzyl]pyrrolidine-1-carboxylate (isomer 2)

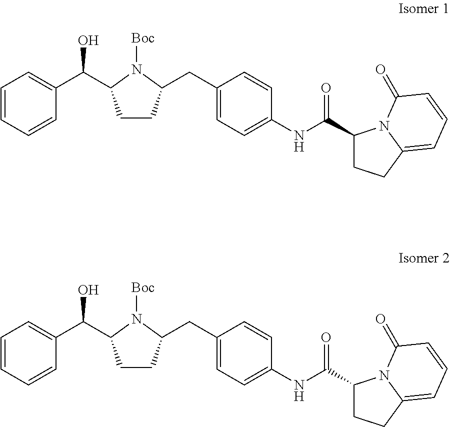

To a solution of 0.610 g (1.60 mmol) of Intermediate i-13a and 0.300 g (1.67 mmol) of Intermediate i-46 in 3.2 mL of anhydrous N,N-dimethylformamide under an atmosphere of nitrogen was added 0.033 g (0.24 mmol) of 1-hydroxy-7-azabenzotriazole followed by 0.336 g (1.75 mmol) of 1-(3-dimethylaminopropyl)-3-ethylcarbodiimide hydrochloride. The resulting suspension was stirred at ambient temperature for 30 min, quenched with water, and extracted with ethyl acetate (3×10 mL). The combined organic layers were washed with brine, dried over magnesium sulfate, filtered and evaporated in vacua. The crude residue was purified by silica gel chromatography eluting with a 50-100% gradient of ethyl acetate in hexanes to afford the title compounds as a mixture of diastereomers in a 97:3 ratio. The two diastereomers were separated by chiral HPLC employing a Daicel CHIRALPAK® AD® column (eluent: 40% IPA in Heptane). The first eluting diastereomer was designated as Isomer 2 and is a colorless solid (0.020 g, 2.3%). LC-MS: m/z (ES) 544.2 (MH)⁺. The second eluting diastereomer was designated as Isomer 1 and is a colorless solid (0.650 g, 75%). LC-MS: m/z (ES) 544.2 (MH)⁺.

Step B (Compound 12): (38)-N-[4-({(2S,5R)-5-[(R)-hydroxy(phenyl)methyl]pyrrolidin-2-yl}methyl)phenyl]-5-oxo-1,2,3,5-tetrahydroindolizine-3-carboxamide

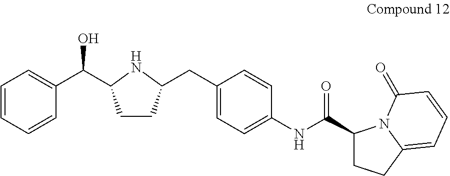

A solution of 0.500 g (0.920 mmol) of Isomer 1 from step A above in 2 mL of isopropanol under an atmosphere of nitrogen was added 4.0 mL of a 4.0 M solution of anhydrous hydrogen chloride in 1,4-dioxane. The reaction mixture was stirred for 1 h and then evaporated to dryness in vacua. The crude reaction mixture was purified by reverse phase HPLC (TMC Pro-Pac C18; 0-75% 0.01% trifluoroacetic acid in acetonitrile/0.01% trifluoroacetic acid in water gradient). The pure fractions were lyophilized overnight then dissolved in a mixture of 10 mL of chloroform and 4 mL of a saturated aqueous bicarbonate solution. The biphasic mixture was stirred vigorously for 10 min, then the layers were separated. The aqueous phase was extracted with chloroform (3×10 mL) and the combined organic layers were washed with brine, dried over magnesium sulfate, filtered and evaporated in vacuo to afford the title compound (Compound 12) as a white solid (0.39 g, 95%). ¹H-NMR (500 MHz, CD₃OD) δ 7.89 (s, 1H), 7.54 (dd, J=8.8, 7.2 Hz, 1H), 7.50 (d, J=8.2 Hz, 2H), 7.34-7.29 (m, 4H), 7.26-7.23 (m, 1H), 7.20 (d, J=8.2 Hz, 2H), 6.38-3.36 (m, 2H), 5.24 (dd, J=9.4, 2.8 Hz, 1H), 4.20 (d, J=7.8 Hz, 1H), 3.35-3.23 (m, 3H), 3.19-3.12 (m, 1H), 2.82-2.71 (m, 2H), 2.60-2.51 (m, 1H), 2.37-2.32 (m, 1H), 1.79-1.72 (m, 1H), 1.52-1.43 (m, 3H). LC-MS: m/z (ES) 444.0 (MH)⁺.

Step B (Compound 13): (3R)-N-[4-({(2S,5R)-5-[(R)-hydroxy(phenyl)methyl]pyrrolidin-2-yl}methyl)phenyl]-5-oxo-1,2,3,5-tetrahydroindolizine-3-carboxamide

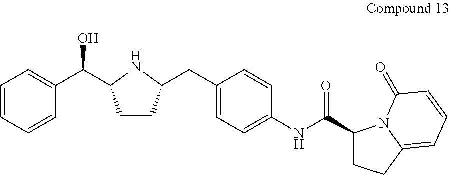

The same procedure was employed for the deprotection of Isomer 2 from Step A above to afford the title compound (Compound 13) as a single diastereomer. LC-MS: m/z (ES) 444.0 (MH)⁺.

Using the Biological Assays as described herein, the human β3 functional activities of Compounds 12 and 13 were determined to be between 1 to 10 nM and less than 1 nM, respectively.

### Compound 14

(6S)-N-[4-({(2S, 5R)-5-[(R)-hydroxy(phenyl)methyl]pyrrolidin-2-yl}methyl)phenyl]-4-oxo-4,6,7,8-tetrahydropyrrolo[1,2-α]pyrimidine-6-carboxamide

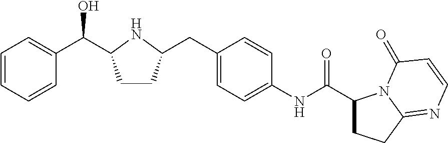

Step A: Tert-butyl(2R,5S)-2-[(R)-hydroxy(phenyl)methyl]-5-[4-({[(6S)-4-oxo-4,6,7,8-tetrahydropyrrolo[1,2-α]pyrimidin-6-yl]carbonyl}amino)benzyl]pyrrolidine-1-carboxylate

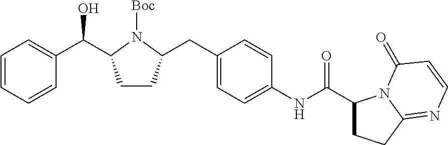

To a solution of i-13a (2L4 g, 55.9 mmol) in N,N-dimethylformamide (100 ml) at 0° C. was added [(6S)-4-oxo-4,6,7,8-tetrahydropyrrolo[1,2-α]pyrimidine-6-carboxylic acid (1-44, 11.1 g, 61.5 mmol), followed by 1-hydroxybenzotriazole (7.55 g, 55.9 mmol), N-(3-dimethylaminopropyl)-N′-ethylearbodiimide hydrochloride (16.1 g, 84.0 mmol) and N,N-diisopropylethylamine (29.2 ml, 168 mmol). The reaction mixture was stirred from 0° C. to ambient temperature for 2 h. Water (600 ml) was added and it was extracted with dichloromethane (600 ml×2). The combined organic layers were dried over Na₂SO₄. After removal of the volatiles, the residue was purified by using a Biotage Horizon® system (0-5% then 5% methanol with 10% ammonia/dichloromethane mixture) to afford the title compound which contained 8% of the minor diastereomer. It was further purified by supercritical fluid chromatography (chiral AS column, 40% methanol) to afford the -title compound as a pale yellow solid (22.0 g, 72%). ¹H NMR (CDCl₃): δ 9.61 (s, 1H), 7.93 (d, J=6.6 Hz, 1H), 7.49 (d, J=8.4 Hz, 2H), 7.35-7.28 (m, 5H), 7.13 (d, J=8.5 Hz, 2H), 6.40 (d, J=6.7 Hz, 1H), 5.36 (d, J=8.6 Hz, 1H), 4.38 (m, 1H), 4.12-4.04 (m, 2H), 3.46 (m,1H), 3.15-3.06 (m, 2H), 2.91 (dd, J=13.1, 9.0 Hz, 1H), 2.55 (m, 1H), 2.38 (m, 1H), 1.71-1.49 (m, 13H). LC-MS 567.4 (M+23).

Step B: (6S)-N-[4-({(2S, 5R)-5-[(R)-hydroxy(phenyl)methyl]pyrrolidin-2-yl}methyl)phenyl]-4-oxo-4,6,7,8-tetrahydropyrrolo[1,2-α]pyrimidine-6-carboxamide

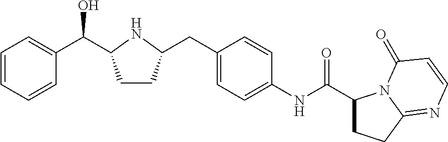

To a solution of the intermediate from Step A (2.50 g, 4.59 mmol) in dichloromethane (40 ml) was added trifluoroacetic acid (15 ml). The reaction mixture was stirred at ambient temperature for 1.5 h. After removal of the volatiles, saturated NaHCO₃ was added to make the PH value to 8-9. The mixture was then extracted with dichloromethane. The combined organic layers were dried over Na₂SO₄. After concentration, crystallization from methanol/acetonitrile afforded the title compound as a white solid (1.23g, 60%). ¹H NMR (DMSO-d₆): δ 10.40 (s, 1H), 7.91 (d, J=6.7 Hz, 1H), 7.49 (d, J=8.3 Hz, 2H), 7.32-7.26 (m, 4H), 7.21 (m,11-1), 7.15 (d, J=8.4 Hz, 2H), 6.23 (d, J=6.7 Hz, 11-1), 5.11 (dd, J=9.6, 2.9 Hz, 1H), 5.10 (br, 1H), 4.21 (d, J=7.1 Hz, 11-1), 3.20-3.00 (m, 4H), 2.66-2.51 (m, 3H), 2.16 (m, 1H), 1.57 (m, 1H), 1.38 (m, 1H), 1.29-1.23 (m, 2H). LC-MS 445.3 (M+1).

Using the Biological Assays as described herein, the human β3 functional activity of Compound 14 was determined to be between 11 to 100 nM.

Compounds 15-29 were prepared using procedures similar to those described above from the appropriate starting materials.

Biological Assays for β3 Functional Activities: The following in vitro assays are suitable for screening compounds that have selective β3 agonist activity:

Functional Assay: cAMP production in response to ligand is measured according to Barton, et al. (1991, Agonist-induced desensitization of D2 dopamine receptors in human Y-79 retinoblastoma cells. Mol. Pharmacol. v3229:650-658) modified as follows. cAMP production is measured using a homogenous time-resolved fluorescence resonance energy transfer immunoassay (LANCE™, Perkin Elmer) according to the manufacture's instructions. Chinese hamster ovary (CHO) cells, stably transfected with the cloned 13-adrenergic receptor (β1, β2 or β3) are harvested after 3 days of subculturing. Harvesting of cells is done with Enzyme-free Dissociation Media (Specialty Media). Cells are then counted and resuspended in assay buffer (Hank's Balanced salt solution supplemented with 5 mM HEPES, 0.1% BSA) containing a phosphodiesterase inhibitor (IBMX, 0.6 mM). The reaction is initiated by mixing 6,000 cells in 6 with 6 μL Alexa Fluor labeled cAMP antibody (LANCE™ kit) which is then added to an assay well containing 12 μL of compound (diluted in assay buffer to 2× final concentration). The reaction proceeds for 30 min at room temperature and is terminated by the addition of 24 detection buffer (LANCE™ kit). The assay plate is then incubated for 1 h at room temperature and time-resolved fluorescence measured on a Perkin Elmer Envision reader or equivalent. The unknown cAMP level is determined by comparing fluorescence levels to a cAMP standard curve.

The non-selective, full agonist 13-adrenergic ligand isoproterenol is used at all three receptors to determine maximal stimulation. The human β3 adrenergic receptor (AR) selective ligand (S)-N-[4-[2-[[2-hydroxy-3-(4-hydroxyphenoxy)propyl]amino]ethyl]-phenyl]-4-iodobenzenesulfonamide is used as a control in all assays. Isoproterenol is titrated at a final concentration in the assay of 10-10 M to 10-5 and the selective ligand (S)-N-[4-[2-[[2-hydroxy-3-(4-hydroxyphenoxy)propyl]amino] ethyl]phenyl]-4-iodobenzenesulfonarnide is titrated at the β3 receptor at concentration of 10-10 M to 10-5 M. Unknown ligands are titrated at all 3 β-adrenergic receptor subtypes at a final concentration in the assay of 10-10 M to 10-5 M to determine the EC₅₀. The EC₅₀ is defined as the concentration of compound that gives 50% activation of its own maximum. Data are analyzed using Microsoft Excel and Graphpad Prism or an internally developed data analysis software package.

Binding Assay: Compounds are also assayed at the β1 and β2 receptors to determine selectivity. All binding assays are run using membranes prepared from CHO cells recombinantly expressing β1 or β2 receptors. Cells are grown for 3-4 days post splitting; the attached cells are washed with PBS and then lysed in 1mM Tris, pH 7.2 for 10 min on ice. The flasks are scraped to remove the cells and the cells then homogenized using a Teflon/glass homogenizer. Membranes are collected by centrifuging at 38,000×g for 15 min at 4° C. The pelleted membranes are resuspended in TME buffer (50 mM Tris, pH 7.4, 5 mM MgCl₂, 2 mM EDTA) at a concentration of 1 mg protein/mL. Large batches of membranes can be prepared, aliquoted and stored at −70° C. for up to a year without loss of potency. The binding assay is performed by incubating together membranes (2-5 μg of protein), the radiolabelled tracer ¹²⁵I-cyanopindolol (¹²⁵I-CYP, 45 pM), 200 μg of WGA-PVT SPA beads (GE Healthcare) and the test compounds at final concentrations ranging from 10-10 M to 10-5 M in a final volume of 200 μL of TME buffer containing 0.1% BSA. The assay plate is incubated for 1 h with shaking at room temperature and then placed in a Perkin Elmer Trilux scintillation counter. The plates are allowed to rest in the Trilux counter for approximately 10 h in the dark prior to counting. Data are analyzed using a standard 4-parameter non-linear regression analysis using either Graphpad Prism software or an internally developed data analysis package. The IC₅₀ is defined as the concentration of the compound capable of inhibiting 50% of the binding of the radiolabelled tracer (¹²⁵I-CYP). A compound's selectivity for the β3 receptor may be determined by calculating the ratio (IC₅₀ β1 AR, β2 AR)/(EC₅₀ β3 AR).

The β3-AR agonist and the antimuscarinic agent can be administered to the patient at a weight ratio of 500:1 to 1:50. In one embodiment, the weight ratio of the β₃-AR agonist and the antimuscarinic agent is 300:1 to 1:10. In another embodiment, the weight ratio is 300:1 to 1:1. In another embodiment, the weight ratio is 150:1 to 1:1. In another embodiment, the weight ratio is 100:1 to 1:1. In yet another embodiment, the weight ratio is 150:1. In still another embodiment, the weight ratio is 100:1.

The combination therapy may further comprise a selective M₂ antagonist in addition to a β3-AR agonist and an antimuscarinic agent.

As used herein, the phrase “selective M₂ antagonist” is a compound which antagonizes muscarinic M₂ subtype at greater than 10-fold selectivity as compared to another muscarinic subtype, for example, M3 subtype. See Delmendo, Br .1″ Pharmacol. 1989 February; 96(2): 457-64, which is incorporated herein by reference in its entirety, for discussions of selective antagonists.

In one embodiment, the selective M₂ antagonist is methoctramine. Methoctramine is a polymethylene tetramine derivative having the following structure:

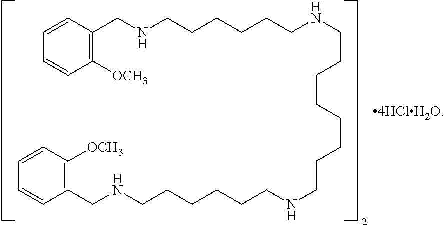

In one embodiment, a method of treating OAB comprises administering to a patient in need thereof a β3-AR agonist, an antimuscarinic agent, and a selective M₂ antagonist. In one embodiment, the antimuscarinic agent has an M₂/M₃ ratio of greater than about 40. In another embodiment, the antimuscarinic agent has an M₂/M₃ ratio of greater than about 50. In one embodiment, the antimuscarinic agent is darifenacin. In one embodiment, the M₂/M₃ ratio is measured using the receptor binding assays described in Ohtake et al.

In one embodiment, a method of treating OAB comprises administering to a patient in need thereof a β3-AR agonist, darifenacin, and methoctramine. In another embodiment, the β3-AR agonist is selected from the compounds shown in Table 3. In another embodiment, the β3-AR agonist is selected from the compounds shown in Table 4. In yet another embodiment, the β3-AR

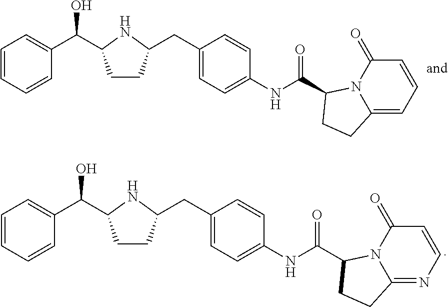

agonist is selected from the group consisting of

In a method wherein a β3-AR agonist, an antimuscarinic agent, and a selective M₂ antagonist are administered to a patient, the β3-AR agonist can be pre-treated with the selective M₂ antagonist. In one embodiment, the selective M₂ antagonist is methoctramine. In another embodiment, the antimuscarinic agent is darifenacin. In another embodiment, the β3-AR agonist is pre-treated with methoctramine. In yet another embodiment, the pre-treated β3-AR agonist with methoctramine is co-administered with darifenacin.

In one embodiment, the concentration of methoctramine for the pre-treatment is 0.1-10 μM. In another embodiment, the concentration of methoctramine for the pre-treatment is 1 μM.

In the combination therapies described above, the β₃-AR agonist, the antimuscarinic agent, and the optional selective M₂ antagonist can be administered to a patient simultaneously, sequentially or separately.

In one embodiment, the β3-AR agonist, the antimuscarinic agent, and the optional selective M₂ antagonist are administered to the patient simultaneously. In another embodiment, the β3-AR agonist, the antimuscarinic agent, and the optional selective M₂ antagonist are administered to the patient separately. In yet another embodiment, β3-AR agonist, the antimuscarinic agent, and the optional selective M₂ antagonist are administered to the patient sequentially.

Suitable patients include, but are not limited to, people with overactive bladder or lower urinary tract symptoms (LUIS). In one embodiment, the patient is a woman with OAB conditions. In another embodiment, the patient is a menopausal woman with OAB conditions.

Another aspect of the present invention provides a combination pharmaceutical composition comprising a β3-AR agonist, an antimuscarinic agent, and an optional selective M₂ antagonist. Suitable β3-AR agonists, antimuscarinic agents, and selective M₂ antagonists are as described above.

Suitable amount of the β3-AR agonist in the combination composition is from about 0.01 mg to abut 500 mg. In one embodiment, the amount of the β3-AR agonist is from about 0.05 mg to abut 250 mg. In another embodiment, the amount is from about 0.1 mg to about 150 mg. In another embodiment, the amount is from about 1 to about 100 mg. In yet another embodiment, the amount is from about 1 to about 50 mg.

Suitable amount of the antimuscarinic agent in the combination composition is from about 0.01 mg to abut 50 mg. In one embodiment, the amount of the antimuscarinic agent is from about 0.05 mg to abut 12 mg. In another embodiment, the amount is from about 0.1 mg to about 6 mg. In another embodiment, the amount is from about 0.2 to about 5 mg. In yet another embodiment, the amount is from about 0.2 to about 3 mg.

Suitable amount of the selective M₂ antagonist is from about 0.01 mg to abut 50 mg. In one embodiment, the amount of the selective M₂ antagonist is from about 0.05 mg to abut 15 mg.

In practical use, the β3-AR agonist, the antimuscarinic agent, and the optional selective M₂ antagonist can be combined as the active ingredients in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are employed. If desired, tablets may be coated by standard aqueous or non-aqueous techniques. Such combination compositions and preparations can contain 0.1-20 percent of each active ingredient. The percentage of active ingredients in these combination compositions may, of course, be varied and the amount of active ingredients in such compositions is such that an effective dosage will be obtained.

The active ingredients can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

The active ingredients may also be administered parenterally. Solutions or suspensions of these active ingredients can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The combination compositions suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

In one embodiment, the combination composition is an oral composition. In another embodiment, the oral composition in a capsule gel. In yet another embodiment, the combination composition is an oral tablet composition. In still another embodiment, the combination composition is an oral bead composition.

In one embodiment, the combination composition is a controlled release composition wherein the antimuscarinic agent is released over 24 hours upon the administration of the composition. In another embodiment, the antimuscarinic agent is released over 10 hours. In yet another embodiment, the antimuscarinic agent is released over 8 hours. In still another embodiment, the antimuscarinic agent is released over 6 hours.

Disclosed herein also include use of a β3-AR agonist, an antimuscarinic agent, and an optional selective M₂ antagonist in the manufacture of a medicament for the treatment or prevention of overactive bladder.

### EXAMPLES

The effects of co-administration of a β3-AR agonist, an antimuscarinic agent, and an optional M₂ antagonist are illustrated in the following examples.

CL 316243, or disodium (R,R)-5-(2-((2-(3-ehlorophenyl)-2-hydroxyethyl)-amino)propyl)-1,3-benzodioxole-2,3-dicarboxylate, is a β3-AR agonist. CL 316243 is described in more detail in J. Med. Chem. 1992 Aug. 7;35(16):3081-4.

Tolterodine, or 2-[(1S)-3-(diisopropylamino)-1-phenylpropyl]-4-methylphenol, is an antimuscarinic agent used to treat overactive bladder. Tolterodine is described in more detail in U.S. Pat. Nos. 5,382,600, 6,630,162, 6,770,295, and 6,911,217.

Oxybutynin, or 4-diethylaminobut-2-ynyl2-cyclohexyl-2-hydroxy-2-phenyl-ethanoate, is an antimuscarinic agent used to relieve urinary and bladder difficulties, including frequent urination and inability to control urination (urge incontinence), by decreasing muscle spasms of the bladder.

Darifenacin, or (S)-2-[1-[2-(2,3-dihydrobenzofuran-5-yl)ethyl] pyrrolidin-3-yl]-2,2-diphenyl-acetamide, is an antimuscarinic agent used to treat urinary incontinence. Darifenacin is described in more detail in U.S. Pat. No. 5,096,890.

### Examples 1-3

### Materials and Methods

The following materials and methods were used for Examples 1-3. Male adult Sprague-Dawley rats were used. After euthanizing using CO₂ gas, a whole bladder was removed. Longitudinal strips (about 6 mm×3 mm) of the extratrigonal portion of the detrusor muscle were prepared. Each strip was placed in a warmed (37° C.) organ bath (25 mL) containing oxygenated (95% O2+5% CO2) Krebs solution. The strips were tied at one end to the organ bath, and connected at the other end to an isometric transducer (AD Instruments) under a resting tension of 10 mN. The responses of the preparations were recorded at a sampling rate of 10 Hz by a multiple channel data acquisition system (PowerLab, AD Instruments), and measured with an analysis software (Chart, AD Instruments). After the equilibration period for at least 60 min, each tissue strip was challenged to electrical field stimulation (EFS) at 60 Hz; duration, 0.3 ms; 3 sec; 90 V to induce contractions. Upon obtaining stable contractions with EFS, compound solution (25 μL) was applied into organ bath in a cumulative manner. After 15 min of each compound treatment, EFS was applied.

### Isobologram Analysis

Isobologram Analysis was used to evaluate the synergic effect of a combination therapy. Isobologram Analysis provides a visual assessment of the interaction of two different agents using independent statistical analysis. The statistical analysis can be accomplished from calculations of certain potency indices from single treatment of each compound and fixed-ratio combinations for the same effect. Isobologram Analysis is described in more detail in WET 298:865-872, 2001, which is incorporated herein by reference in its entirety.

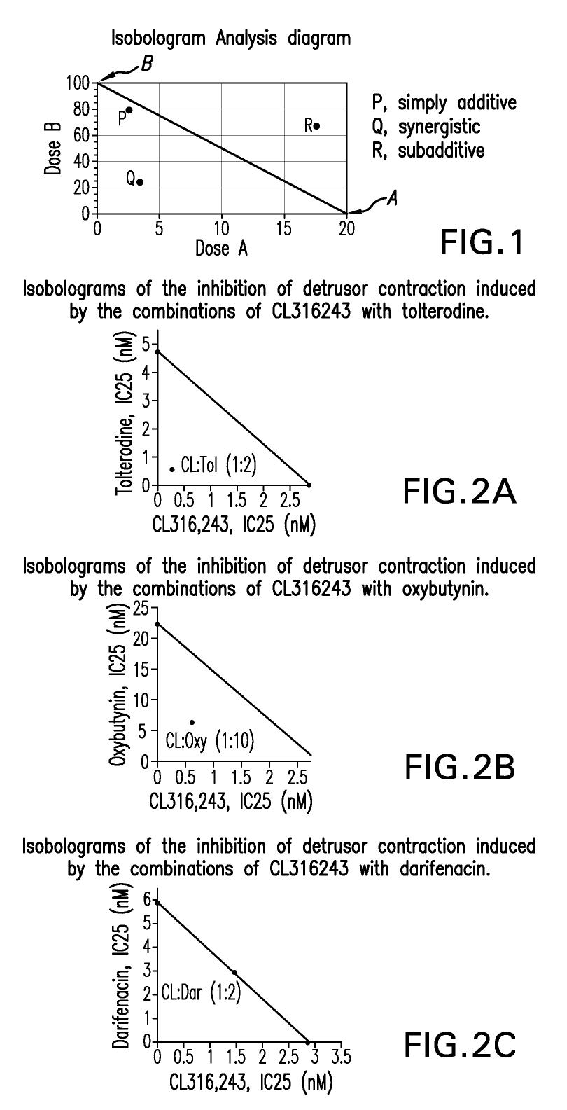

An illustrative diagram of Isobologram Analysis is shown in FIG. 1. In FIG. 1,

Isobologram for some particular effect (e.g., 50% of the maximum) in which the dose of active agent A alone is A=20 and active agent B alone is B=100. The straight line connecting these intercept points (additivity line) is the locus of all dose pairs that, based on these potencies, should give the same effect, An actual dose pair such as point Q attains this effect with lesser quantities and is synergistic (or super-additive), while the dose pair denoted by point R means greater quantities are required and is therefore sub-additive. A point such as P that appears close to the A-B line is simply additive. A suitable statistical analysis is often used to demonstrate the nature of the interaction.

### Example 2

Combination Therapy of β3-AR Agonist CL316243 with an Antimuscarinic Agent Selected from Tolterodine, Oxybutynin, and Darifenacin

When administered individually, each of CL316243, tolterodine, oxybutynin and darifenacin inhibited the EFS-induced isolated detrusor muscle contractions. Table 5 shows the concentration of each compound which induced 25% inhibition. These values were used for the following isobologram analyses.

TABLE 5
Inhibition of detrusor contraction with
CL316243, tolterodine, oxybutynin and
darifenacin

| Compound | IC₂₅ (nM) |
|---|---|
| β3-AR agonist | |
| CL316,243 | 2.86 |
| Antimuscarinic | |
| Tolterodine | 4.71 |
| Oxybutynin | 22.28 |
| Darifenacin | 5.84 |

In the combination therapy, CL316243 was co-administered with tolterodine, oxybutynin or darifenacin at fixed weight ratios and the results from isobologram analyses are shown in FIG. 2. FIG. 2 indicates that combinations of CL316243 with tolterodine (1:2, FIG. 2A) or oxybutynin (1:10, FIG. 2B) showed synergistic effects. On the other hand, the combination of CL316243 with darifenacin (1:2, FIG. 2C) appears to be simply additive (i.e., no synergistic effect).

While not wishing to be bound by theory, it is generally believed that M3 antagonistic activity of an antimuscarinic agent is important for OAB efficacy (see, for example,

Abrams and Andersson. BTU Int, 100, 987-1006 (2007)). It has now surprisingly been found that the relative selectivity of M₂/M₃ of the antimuscarinic agent may play an important role for the OAB efficacy and/or reduced side effects in the combination therapy using the antimuscarinic agent and a β3-AR agonist.

As can be seen from the results above, antimuscarinic agent tolterodine has about equal selectivity on M₂ and M₃ subtypes of the muscarinic receptors (M₂/M₃≠1), and the combination of tolterodine and CL316243, a β3-AR agonist, at 2:1 ratio provided synergistic effect. Another antimuscarinic agent oxybutynin, which has M₂/M₃ ratio of about 6, also provided synergistic effect when combined with CL316243 at 10:1 ratio.

On the other hand, darifenacin which has much higher selectivity for M₃ than M₂ (M₂/M₃≠50), did not provide synergy when combined with CL316243 at 2:1 ratio.

In summary, the combination of CL316243 with tolterodine or oxybutynin, each of which has M₂/M₃ ratio of less than 40, provided synergy in the inhibition of detrusor contraction. On the other hand, the combination of CL316243 with darifenacin, which has M₂/M₃ ratio of greater than 40, did not provide synergy.

### Example 3

β3-AR Agonist Compound 12 in Combination with Tolterodine or Darifenacin

In this example, a different β3-AR Agonist was used to study the synergistic effect of the combination therapy of a β3-AR Agonist and an antimuscarinic agent.

The β3-AR agonist Compound 12 described above in Table 3 inhibited the EFS-induced isolated detrusor muscle contractions with an IC₂₅ value of 275 nM. Thus Compound 12 is nearly 100 times less potent than CL316243 (IC₂₅ 2.86 nM, see Table 5) in inhibiting the EFS-induced contraction of rat bladder strips. This is consistent with less potent activity of Compound 12 at rat β3-AR.

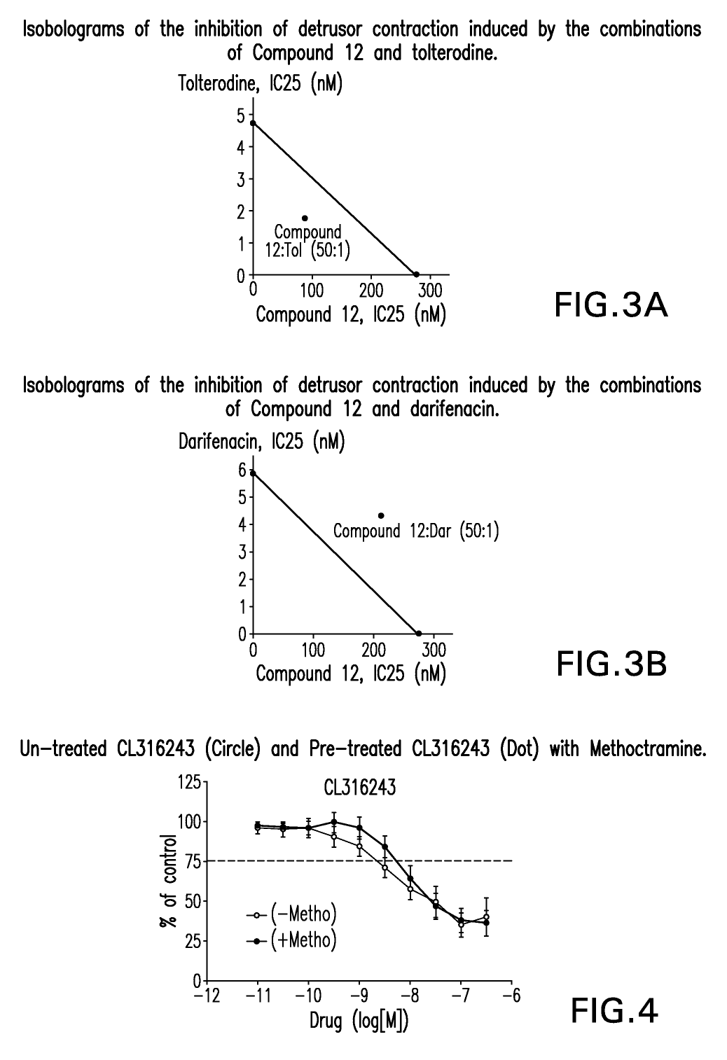

In the combination study, Compound 12 was co-administered with tolterodine or darifenacin at a fixed weight ratio of 50:1. The isobologram analyses results are shown in FIG. 3. FIG. 3 indicates that the combination of Compound 12 with tolterodine, which has M₂/M₃ of about 1, at 50:1 ratio (FIG. 3A), provided synergistic effect.

On the other hand, the combination of Compound 12 with darifenacin, which has M₂/M₃ of about 50, at 50:1 ratio (FIG. 3B), did not provide synergistic effect (sub-additive).

The above results are consistent with the results observed in Example 1, where a different β3-AR agonist (CL316243) was used in the studies. These results suggest that when the antimuscarinic agent has M₂/M₃ ratio of less than 40, its combination with a β3-AR agonist provided synergy in the inhibition of detrusor contraction. On the other hand, when the antimuscarinic agent has M₂/M₃ ratio of greater than 40, its combination with a β3-AR agonist provided no synergy.

### Example 4

### Effect of Selective M₇ Antagonist on the Synergistic Effect of the Combination Therapy

In this example, the effect of a selective M₂ antagonist on the combination therapy of a β3-AR agonist and an antimuscarinic agent having M₂/M₃ ratio of greater than 40 is studied.

First, CL 316243, a β3-AR agonist, was either un-treated or pre-treated with methoctramine (1 μM) and the results are shown in FIG. 4. FIG. 4 shows that pretreatment of CL316243 with methoctramine did not significantly affect the potency of CL316243 with regard to inhibiting the EFS-induced bladder contraction. This result is consistent with the general belief that a selective M₂ antagonist alone does not relax pre-contracted rat bladder strips as do β3-AR agonists and antimuscarinics with M₃ antagonism. This suggests that the combination of a selective M₂ antagonist and CL316243, and without the presence of M₃ antagonism, did not provide synergism.

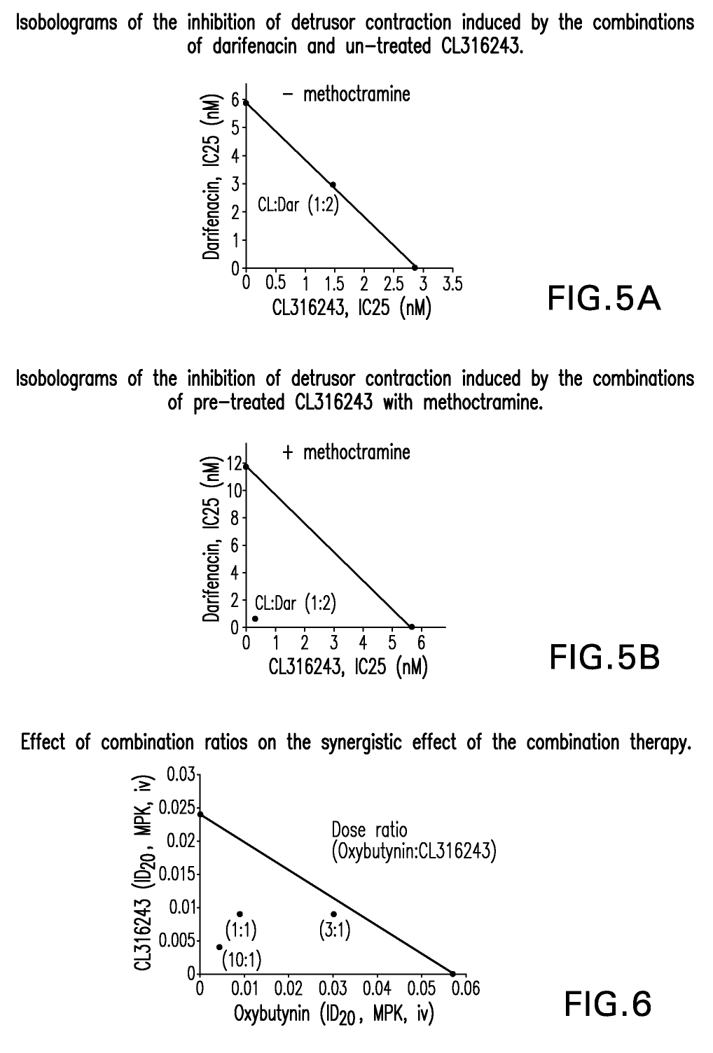

Next, each of the un-treated (A) and pre-treated CL316243 (B) was combined with darifenacin, respectively, at a ratio of 1:2 and the results are shown in FIG. 5. FIG. 5 indicates that the combination of pre-treated CL316243 (with 1 μM methoctramine) and darifenacin at 1:2 ratio provided synergistic effect. As discussed above, darifenacin is a selective M₃ antagonist and has M₂/M₃ ratio of about 50.

On the other hand, the combination of the un-treated CL316243 and darifenacin at the same ratio (1:2) was simply additive (i.e., no synergistic effect).

These results are consistent with the observations in Examples 1 and 2 that the synergistic effect of the combination between a β3-AR agonist and an antimuscarinic agent may require the presence of both M₂ and M₃ antagonism.

While not wishing to be bound by theory, it is believed that M₂ receptors may play a role in mediating an indirect contractile response by reversing adrenoceptor-mediated relaxation through a cAMP-dependent mechanism. M₂ antagonism may potentiate β3-AR agonist induced cAMP increase and BK channel opening, resulting in further relaxation of detrusor muscle.

### Example 5

### Effect of Combination Ratios on the Synergistic Effect

### Materials and Methods

Animals: female SD rats (200-250 g BW) (Seven groups in total).
Anesthesia: urethane (1.0 g/kg, ip).
Parameter: amplitude of distention-induced rhythmic bladder contraction.
Compounds: oxybutynin (OXY), CL 316243 (CL).
Analysis: isobologram using ID20 values (doses decreasing amplitude by 20%).

First, single dosing study was conducted to calculate ID20 values of oxybutynin (OXY) and CL316243 (CL) at the following conditions and the ID20 values obtained are shown in Table 6:
Vehicle (saline);
OXY, 0.01, 0.03, 0.1 mg/kg, iv;
CL, 0.003, 0.01, 0.03 mg/kg, iv.

TABLE 6
ID20 values of CL316243
and Oxybutynin.

| | ID20 |
|---|---|
| Compound | (MPK, iv)- |
| Oxybutynin | 0.057 |
| CL316243 | 0.024 |

Results in Table 6 show that both oxybutynin and CL316243 decreased the amplitude of distention-induced rhythmic bladder contraction in anesthetized female rats.

Next, combination dosing regimens shown below in Table 7 were conducted to compose an isobologram. There were 9 groups in total. ID20 values of OXY and CL was calculated in each combination rate.

TABLE 7
Dosing Regimens (mg/kg).

| OXY:CL | 1:1 | 10:1 | 3:1 |
|---|---|---|---|
| Regimen 1 | 0.003:0.003 | 0.03:0.003 | 0.01:0.003 |
| Regimen 2 | 0.01:0.01 | 0.1:0.01 | 0.03:0.01 |
| Regimen 3 | 0.03:0.03 | 0.3:0.03 | 0.1:0.03 |

FIG. 6 indicates that synergistic effects were observed for combinations of CL and OXY at 1:1 and 1:10 ratios. On the other hand, the combination of CL and OXY at 1:3 only provided a simple additive, but not synergistic, effect.

These results suggest that the synergistic effect between the β3-AR agonist CL and the antimuscarinic agent OXY also depends on the particular combination ratio in the combination.

### Example 6

### Combination Composition Comprising β3-AR Agonist and Antimuscarinic Agent

An exemplary combination composition comprising a β3-AR agonist and an antimuscarinic agent is shown in Table 8:

TABLE 8
Combination Composition of a β3-AR
agonist and an antimuscarinic Agent

| | |
|---|---|
| Ingredient ID | Composition, wt % |

CR formulation

| | |
|---|---|
| Antimuscarinic agent | 0.01-5 |
| β3-AR agonist | 0.1-10 |
| Filler | 10-95 |
| Binder | 0.1-10 |
| Lubricant | 0.1-5 |
| CR coating | 0.5-20 |
| Coloring agent | 0.1-10 |

Note:
The weight percentage (wt %) in table 8 is based on the total weight of the combination composition.

In one embodiment, the β3-AR agonist is selected from the compounds listed in Table 3. In another embodiment, the antimuscarinic agent is selected from tolterodine, fesoterodine, oxybutynin, solifenacin, propiverin, trospium, imidafenacin, and TD6301.

In one embodiment, the above combination composition is a controlled release (CR) formulation. In another embodiment, the combination composition is in a capsule gel for oral administration.

### Example 7

### Combination Composition Comprising CR Antimuscarinic Agent and IR β3-AR Agonist

An exemplary combination composition comprising an antimuscarinic agent in a controlled release (CR) portion and a β3-AR agonist in an immediate release (CR) portion is shown in Table 9:

TABLE 9
Combination Composition of a β3-AR
agonist and an antimuscarinic Agent

| | |
|---|---|
| | Composition, |
| Ingredient ID | wt % |

CR Beads of antimuscarinic agent

| | |
|---|---|
| Antimuscarinic agent | 0.01-5 |
| Filler | 1-95 |
| Binder | 0.1-10 |
| Lubricant | 0.1-5 |
| CR coating | 0.5-20 |
| Coloring agent | 0.1-10 |

IR Beads of β3-AR agonist

| | |
|---|---|
| β3-AR agonist | 0.1-10 |
| Filler | 1-95 |
| Binder | 0.1-10 |
| Lubricant | 0.1-5 |
| IR coating | 0.5-20 |
| Coloring agent | 0.1-10 |

Note:
The weight percentage (wt %) in table 9 is based on the total weight of each respective
portion of the combination composition.

In one embodiment, the β3-AR agonist is selected from the compounds listed in Table 3. In another embodiment, the antimuscarinic agent is selected from tolterodine, fesoterodine, oxybutynin, solifenacin, propiverin, trospium, imidafenacin, and TD6301.

In one embodiment, the above composition is in a capsule gel for oral administration.

### Example 8

### Effect of Combination Therapy on Bladder Capacity in Rhesus Monkeys

### Materials and Methods

Adult female rhesus monkeys (Macaca mulatta) weighed 5.3-6.2 kg (4-7 year-old) were used. The subjects were either paired or individually housed on a 12-h light/12-h dark cycle (lights on at 7:00 AM). Their diet consisted of 2050 Teklad (Harlan Laboratories, Indianapolis, Ind.) and fresh fruit or vegetable. Water was freely available. All animals were observed daily by a veterinary technician and caretakers for signs of ill health. Subjects were repeatedly used with >13-day resting period. Monkeys were anesthetized with an intramuscular injection of either Telazol (3-5 mg/kg) or ketamine (10-20 mg/kg) followed by intravenous constant rate infusion with ketamine (0.2-0,8 mg/kg/min) using a syringe pump (552222, Harvard Apparatus, Holliston, Mass.). Animals were placed in a supine position and a triple lumen balloon transurethral catheter (7.4 Fr, Cook Medical, Bloomington, Ind.) was inserted into the bladder and the balloon was inflated with 1 mL of water to secure the tip of catheter at the bladder base. The catheter was connected with an infusion pump (Gemini PC-2TX, ALARIS Medical Systems, San Diego, Calif.) for bladder filling and a pressure transducer for intravesical pressure monitoring. Intravesical pressure was continuously recorded using a multiple channel data acquisition system (Power lab, AD Instruments, Biopac systems, Colorado Springs, Colo.) at a sampling rate of 20 Hz. After confirming bladder emptiness by an ultrasonography (Logiq e vet, GE Medical Systems, Waukesha, Wis., FIG. 1A), saline was intravesically infused at 15 mL/min. When the steep rise in pressure indicative of the micturition reflex was observed, intravesical infusion was stopped and the bladder was manually emptied with a 60 ml syringe.

After two baseline cystometry readings, drug was intravenously administered three times using a rising dose paradigm with a cystometry performed 10 min after each dose. Bladder capacity was measured for each cystometry and % change from the baseline capacity was calculated. As used herein, the term “baseline capacity” or “baseline” means the average bladder capacity from two pre-dose measurements.

Mono-therapies using either tolterodine (“TOL”), darifenacin (DAR”) or Compound 14 (Cpd 14”) at various doses and combination therapies of TOL:Cpd 14 and DAR:Cpd 14 at different doses and dose ratios as shown in Table 10 were tested in rhesus monkeys.

TABLE 10
Doses and Dose Ratios of Mono-therapies
and Combination Therapies

| | | Dose Ratio | | | | | | |
|---|---|---|---|---|---|---|---|---|
| | | Compound 14 (mg/kg) | | | | | | |
| | | 0 (ve- | | | | | | |
| | Dose | hicle) | 0.003 | 0.01 | 0.03 | 0.1 | 0.3 | 1 |
| Tolter- | 0 (ve- | Vehicle | Mono | Mono | Mono | Mono | Mono | Mono |
| odine | hicle) | | | | | | | |
| (mg/kg) | 0.01 | Mono | 3.3:1 | 1:1 | | | | |
| | 0.03 | Mono | 10:1 | 3:1 | 1:1 | | | |
| | 0.1 | Mono | | | 3.3:1 | 1:1 | | 1:10 |
| Dari- | 0.01 | Mono | | 1:1 | 1:3 | | | |
| fenacin | 0.03 | Mono | | | 1:1 | 1:3.3 | | |
| (mg/kg) | 0.1 | Mono | | | | 1:1 | 1:3 | |

Using the above mono-therapies and combination therapies, the bladder capacity results in rhesus monkeys are summarized in Table 11. The reported results are mean values of % change from baseline in 4-6 animals.

TABLE 11
Bladder Capacity Results Using Mono-therapies
and Combination Therapies in Rhesus Monkeys

| | | Bladder Capacity as Measured in % Change from Baseline | | | | | | |
|---|---|---|---|---|---|---|---|---|
| | | Compound 14 (mg/kg) | | | | | | |
| | | 0 (vehicle) | 0.003 | 0.01 | 0.03 | 0.1 | 0.3 | 1 |
| TOL | 0 (vehicle) | −0.13% | 4.1% | 18.0% | 27.3% | 33.9% | 31% | 55.9% |
| (mg/kg) | 0.01 | 8.6% | 28.2% | 31.0% | | | | |
| | 0.03 | 16.8% | 35.5% | 36.4% | 43.5% | | | |
| | 0.1 | 40.3% | | | 62.9% | 56.8% | | 69.6% |
| DAR | 0.01 | 8.9% | | 13.7% | 23.7% | | | |
| (mg/kg) | 0.03 | 18.3% | | | 37.1% | 43.3% | | |
| | 0.1 | 29.4% | | | | 58.0% | 68.7% | |

It can be seen from Table 11 that all combinations of Compound 14 and tolterodine tested showed greater bladder capacity as compared to each respective mono-therapy. It is to be noted that at the lowest dose of compound 14 (0.003 mg/kg), the synergistic effects of the combinations were much larger. Specifically, the combination of Cpd 14:TOL at 0.003 mg/kg:0.01 mg/kg showed bladder capacity increase of 28.2% as compared to 4.1% and 8.6% for the respective mono-therapies. Similarly, the combination of Cpd 14:TOL at 0.003 mg/kg:0.03 mg/kg showed bladder capacity increase of 35.5% as compared to 4.1% and 16.8% for the respective mono-therapies.

For combination therapies of Compound 14 and darifenacin, combinations showed superior bladder capacity effect at higher doses of darifenacin (0.03, 0.1 mg/kg).

Tolterodine, a non-selective muscarinic antagonist, clearly demonstrated improved efficacy with Compound 14 at the examined combinations, while additional efficacy with darifenacin, a selective M3 antagonist, and Compound 14 combinations was limited only at higher doses. These results suggest that both M2 and M3 antagonism of an antimuscarinic agent may be important for improved efficacy when combined with a β3-AR agonist.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A method of treating overactive bladder, wherein the method comprises administering to a patient in need thereof:
a β3-AR agonist,
an antimuscarinic agent, and
an optional selective M₂ antagonist;
wherein the β3-AR agonist is selected from the group consisting of:

| Compound # | Structure |
|---|---|
| 1 | 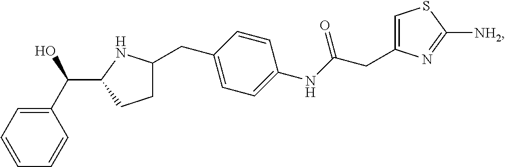 |
| 2 | 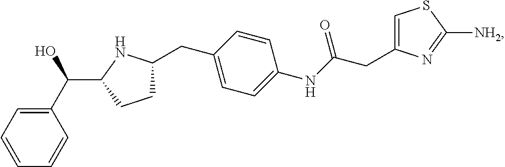 |
| 3 | 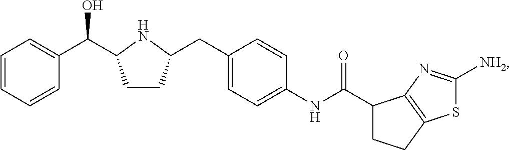 |
| 4 | 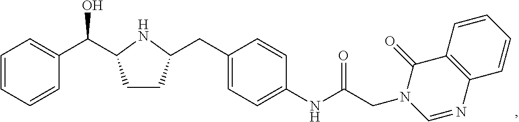 |
| 5 | 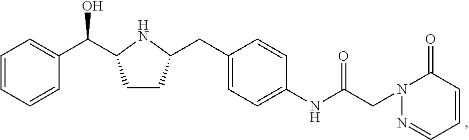 |
| 6 | 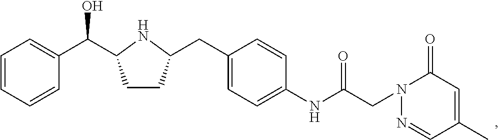 |
| 7 | 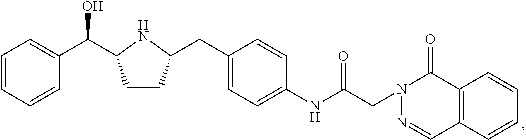 |
| 8 | 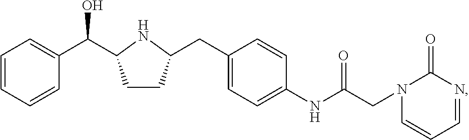 |
| 9 | 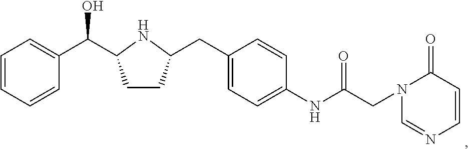 |
| 10 | 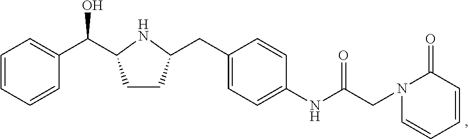 |
| 11 | 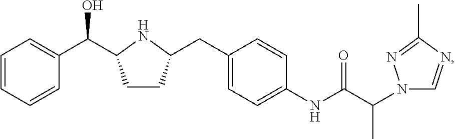 |
| 12 | 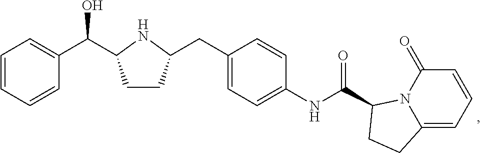 |
| 13 | 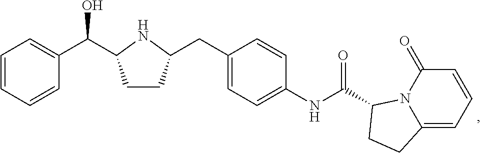 |
| 14 | 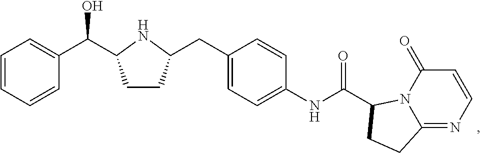 |
| 15 | 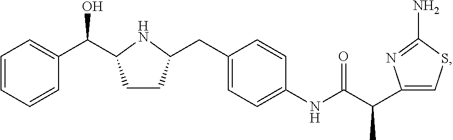 |
| 16 | 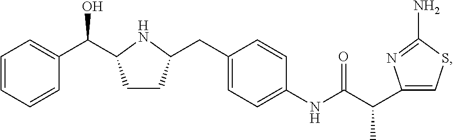 |
| 17 | 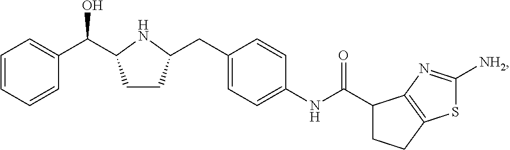 |
| 18 | 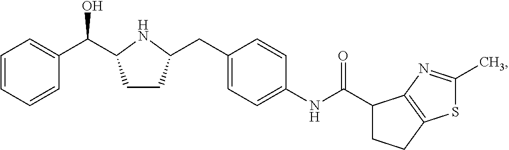 |
| 19 | 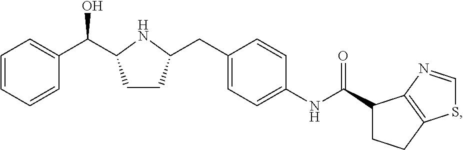 |
| 20 | 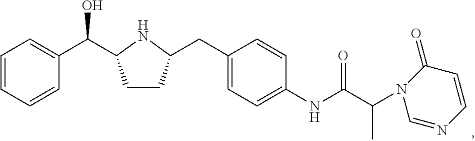 |
| 21 | 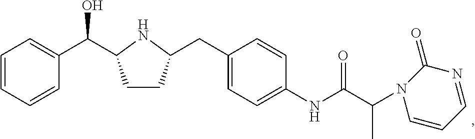 |
| 22 | 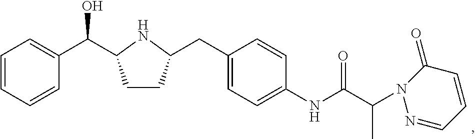 |
| 23 | 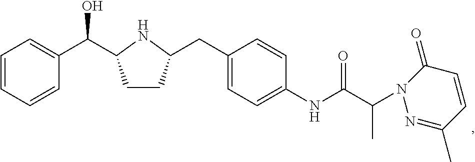 |
| 24 | 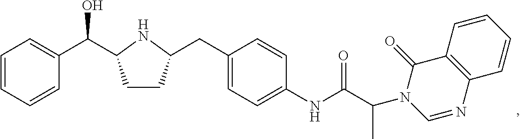 |
| 25 | 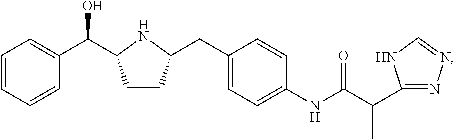 |
| 26 | 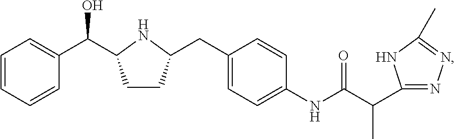 |
| 27 | 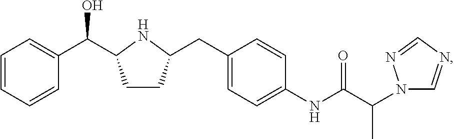 |
| 28 | 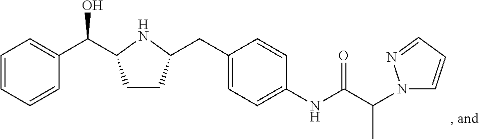 |
| 29 | 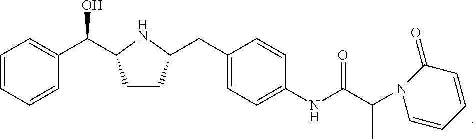 |

2. The method of claim 1, wherein the antimuscarinic agent has an M₂/M₃ ratio of less than 40.

3. The method of claim 2, wherein the antimuscarinic agent has an M₂/M₃ ratio of less than 20.

4. The method of claim 2, wherein the antimuscarinic agent is selected from the group consisting of: tolterodine, fesoterodine, oxybutynin, solifenacin, propiverin, trospium, imidafenacin, and TD6301.

5. The method of claim 4, wherein the antimuscarinic agent is tolterodine or oxybutynin.

6. The method of claim 1, wherein the β3-AR agonist is selected from the group consisting of:

| Compound # | Structure |
|---|---|
| 11 | 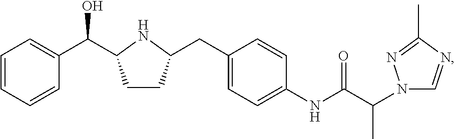 |
| 12 | 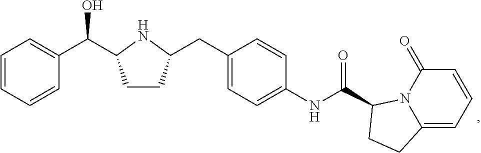 |
| 13 | 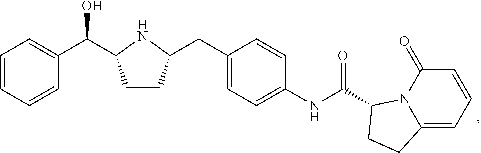 |
| 14 | 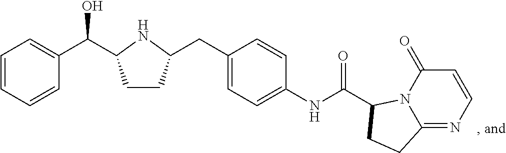 |
| 26 | 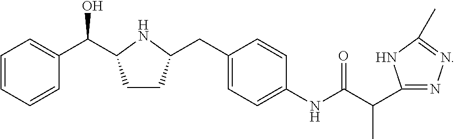 |

7. The method of claim 6, wherein the β3-AR agonist and the antimuscarinic agent are administered to the patient at a weight ratio of 300:1 to 1:10.

8. The method of claim 6, wherein the antimuscarinic agent is tolterodine, and wherein the β3-AR agonist and tolterodine are administered to the patient at a weight ratio of 300:1 to 1:1.

9. The method of claim 1, wherein the method comprises administering to the patient:
a β3-AR agonist,
an antimuscarinic agent, and
a selective M₂ antagonist.

10. The method of claim 9, wherein the antimuscarinic agent has an M₂/M₃ ratio of greater than 40.

11. The method of claim 10, wherein the antimuscarinic agent is darifenacin and the selective M₂ antagonist is methoctramine.

12. A method of treating overactive bladder, wherein the method comprises administering to a patient in need thereof:
CL316243, and
oxybutynin;
wherein CL316243 and oxybutynin are administered to the patient at a weight ratio of 1:1 or 1:10.

13. The method of claim 1, wherein the β3-AR agonist, the antimuscarinic agent, and the optional selective M₂ antagonist are administered simultaneously, separately or sequentially.

14. The method of claim 1, wherein the β3-AR agonist, the antimuscarinic agent, and the optional selective M₂ antagonist are administered orally.

15. A pharmaceutical composition comprising:
a β3-AR agonist,
an antimuscarinic agent, and
an optional selective M₂ antagonist;
wherein the β3-AR agonist is selected from the gratin consisting of:

| Compound # | Structure |
|---|---|
| 11 | 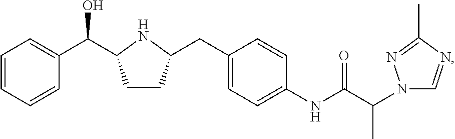 |
| 12 | 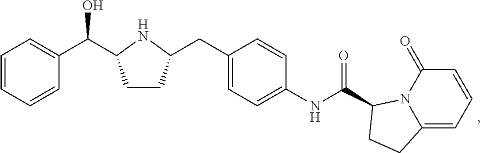 |
| 13 | 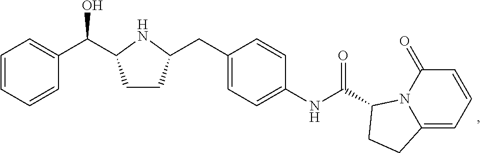 |
| 14 | 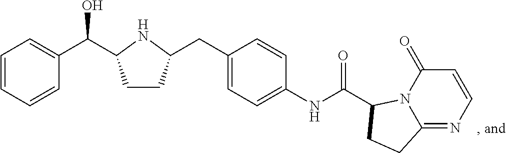 |
| 26 | 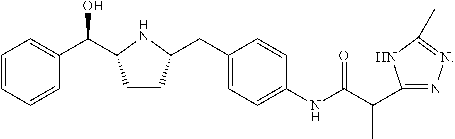 |

16. The pharmaceutical composition of claim 15, wherein the composition comprises:
a β3-AR agonist, and
an antimuscarinic agent; and
wherein the antimuscarinic agent has an M₂/M₃ ratio of less than 40.

17. The pharmaceutical composition of claim 16, wherein the antimuscarinic agent is selected from the group consisting of: tolterodine, oxybutynin, fesoterodine, solifenacin, propiverine, and trospium.

18. The pharmaceutical composition of claim 15, wherein the composition comprises:
a β3-AR agonist,
an antimuscarinic agent, and
a selective M₂ antagonist;
wherein the antimuscarinic agent is darifenacin, and
wherein the selective M₂ antagonist is methoctramine.

19. The pharmaceutical composition of claim 15, wherein the composition is a tablet or capsule for oral administration.

20. The pharmaceutical composition of claim 15, wherein the composition provides controlled release of the antimuscarinic agent.
